(19) United States
(12) Patent Application Publication
(10) Pub. No.: US 2026/0234468 A1
(43) Pub. Date: Aug. 13, 2026

(54) FLUORESCENT NON-CONJUGATED POLYMER NANOPARTICLE WITH TANDEM DYES

(71) Applicant: Cytek Biosciences, Inc., Fremont, CA (US)

(72) Inventors: Yu Rong, Fremont, CA (US); Ivette Amador, Fremont, CA (US); Xingyoung Wu, Fremont, CA (US); Alive Wang, Fremont, CA (US); Rong Zhang, Fremont, CA (US); Su Chen, Fremont, CA (US); Jun Deng, Fremont, CA (US); Ming Yan, Fremont, CA (US)

(73) Assignee: Cytek Biosciences, Inc., Fremont, CA (US)

(21) Appl. No.: 19/432,869

(22) Filed: Dec. 24, 2025

Related U.S. Application Data
(60) Provisional application No. 63/738,715, filed on Dec. 24, 2024.

(51) Int. Cl. C09K 11/06; G01N 15/14; G01N 33/533; G01N 33/58

(52) U.S. Cl. CPC C09K 11/06; G01N 15/1459; G01N 33/533; G01N 33/582

(57) ABSTRACT

Embodiments of a fluorescent non-conjugated polymer tandem dye nanoparticle is disclosed. In some embodiments, the fluorescent non-conjugated polymer tandem dye nanoparticle comprise a non-conjugated polymer backbone coupled to one or more donor monomer, FRET monomer, acceptor dye, and functional group. In some embodiments, the donor, FRET monomer, and functional group are formed into nanoparticles and the acceptor dye is grafted on after formation of the nanoparticle. In other embodiments, the donor and functional group form nanoparticles, the acceptor dye is grafted on after formation of the nanoparticle.

Antibodies can be bioconjugated to the fluorescent non-conjugated polymer tandem dye nanoparticle for use in full spectrum flow cytometry.

### CROSS-REFERENCE TO RELATED APPLICATIONS

This patent application claims the benefit and is non-provisional of U.S. Provisional Patent Application No. 63/738,715 titled FLUORESCENT NONCONJUGATED POLYMER NANOPARTICLE WITH TANDEM DYES filed on Dec. 24, 2024, by inventors Yu Rong et al., incorporated herein by reference for all intents and purposes.

This patent application is related to U.S. Provisional Patent Application No. 63/521,692 titled ULTRA-BRIGHT NANOPARTICLE FLUORESCENT DYE COMPLEXES filed on Jun. 18, 2023, by inventors Yu Rong et al., incorporated herein by reference for all intents and purposes. This patent application is related to U.S. Non-Provisional patent application Ser. No. 17/304,843 titled METHODS OF FORMING MULTI-COLOR FLUORESCENCE-BASED FLOW CYTOMETRY PANEL filed on Jun. 26, 2021, by inventors Maria Jaimes et al., incorporated herein by reference for all intents and purposes. U.S. Non-Provisional patent application Ser. No. 17/304,843 claims the benefit of U.S. Provisional Patent Application No. 63/045,040 titled METHODS OF FORMING MULTI-COLOR FLUORESCENCE-BASED FLOW CYTOMETRY PANEL filed on Jun. 26, 2020, by inventors Maria Jaimes et al., incorporated herein by reference for all intents and purposes. U.S. Non-Provisional patent application Ser. No. 17/304,843 also claims the benefit of U.S. Provisional Patent Application No. 63/045,103 titled METHODS OF FORMING MULTI-COLOR FLUORESCENCE-BASED FLOW CYTOMETRY PANEL filed on Jun. 27, 2020, by inventors Maria Jaimes et al., incorporated herein by reference for all intents and purposes.

This patent application is further related to U.S. Patent Application No. Ser. No. 15/659,610 titled COMPACT DETECTION MODULE FOR FLOW CYTOMETERS filed on Jul. 25, 2017, by inventors Ming Yan et al., incorporated herein by reference for all intents and purposes. This patent application is further related to U.S. patent application Ser. No. 15/498,397 titled COMPACT MULTI-COLOR FLOW CYTOMETER filed on Apr. 26, 2017, by David Vrane et al. that describes a flow cytometer with which the embodiments can be used and is incorporated herein by reference for all intents and purposes. This patent application is further related to U.S. patent application Ser. No. 16/418,942 titled FAST RECOMPENSATION OF FLOW CYTOMETERY DATA FOR SPILLOVER READJUSTMENTS filed on May 21, 2019, by Zhenyu Zhang that describes matrices with which the embodiments can be used and is incorporated herein by reference for all intents and purposes.

### FIELD

The embodiments of the invention relate generally to methods to synthesize fluorescent non-conjugated polymer nanoparticles with tandem dyes for flow cytometry and other biological applications.

### BACKGROUND

Flow cytometry is a technology that provides rapid multi-parametric analysis of single cells in solution. Flow cytometers utilize lasers as light sources to produce both scattered and fluorescent light signals that are read by detectors such as photodiodes or photomultiplier tubes. These signals are converted into electronic signals that are analyzed by a computer and written to a data file. Cell populations can be analyzed and/or purified based on their fluorescent or light scattering characteristics.

Flow cytometry generally involves a sample containing cells or particles suspended in a fluid and injected into the flow cytometer instrument. The fluid containing the sample is focused to ideally allow the sample to flow one cell at a time through a laser beam, where the light scattered is characteristic to the cells and their components. Cells are often labeled with fluorescent markers or fluorochromes, so light is absorbed and then emitted in a band of wavelengths. Tens of thousands of cells can be quickly examined and the data gathered are processed by a computer

Fluorochromes are excited over a wavelength range (excitation wavelength range) associated with the wavelength of the laser and when excited, can emit fluorescence over a different wavelength range (emission wavelength range). The wavelength range of each detector module is associated with the expected emission wavelength range from the excitation of fluorochromes for the associated laser.

Flow cytometry is a powerful tool that has applications in immunology, molecular biology, bacteriology, virology, cancer biology and infectious disease monitoring allowing unprecedented detail in studies of the immune system and other areas of cell biology.

A full spectrum flow cytometer measures the entire fluorochrome emission, from ultra-violet to near infra-red, across multiple lasers using many more detectors compared to a conventional flow cytometer. It produces very specific spectral fingerprints that are used to mathematically distinguish one fluorophore from another, even when their maximum emissions (the primary component measured by a conventional flow cytometer) are very similar. Leveraging this full spectrum technology, the ability to combine thirty or more fluorescently labeled antibodies becomes possible using a fluorescence-based full spectrum flow cytometer.

Fluorescence detection of marked cells is important in flow cytometry, cell sorting, and other biological experiments with different laboratory equipment. Improvement in the markers (dyes) that are excited by lasers to give off fluorescent light (fluorescence) can help better identify the cells mixed into a biological sample of multiple differing cells with different cell types. Excitation of a marker can be improved by a more efficient absorption by a dye molecule of an excitation light which can lead to improved fluorescence intensity and its detection. If the number of dye molecules excited by the same laser spot beam can be increased, then the intensity of the fluorescence light can be increased to improve fluorescence detection.

Fluorescence imaging is a most widely used modality for clinical studies and biological research due to its advantages, including good spatial and temporal resolution, high sensitivity and selectivity, minimal invasiveness, and excellent tunability, so several strategies for developing brighter fluorescent probes have been pursued in the past. Green fluorescent proteins (GFP) [Yu, J.; Xiao, J.; Ren, X. J.; Lao, K. Q.; Xie, X. S.; Science 2006, 311, 1600 1603.], Quantum dots (QDs) [Mingyong Han; Xiaohu Gao; Jack Z. Su; and Shuming Nie; nature biotechnology, 2001, 19, 631-635], Carbon nano-dots (C-dots) [Sheila N. Baker, Gary A.; Chem. Int. Ed. 2010, 49, 2-21.], nano-diamonds [V Vaijayanthimala, H-C Chang; Nanomedicine (2009) 4(1), 47-55] and other nano-materials broaden researchers' tools for watching biology besides fluorescent dyes.

Among fluorescent polymers, fluorescent conjugated polymers have recently attracted considerable attention and have been extensively studied for both optoelectronic and biological applications in past few decades. Fluorescent conjugated polymer in different shapes: conjugated polymer nanoparticles (Pdots) [CHIU, Daniel, T.; WU, Chang-feng; RONG, Yu; ZHANG, Yong; WU, Yi-Che; CHAN, Yang-Hsiang; ZHANG, Xuanjun; YU, Jiangbo; SUN, Wei; WO 2013/101902 A2] and conjugated polymer polyelectrolyte [Glenn P. Bartholomew; Yongchao Liang, US 2018/0163054 A1] [XU, Xinshe; WANG, Jing; YEROU, Matthew, WO 2019/023463 A1] have been commercialized in the past 10 years. However, it is still challenging to synthesize structurally perfect conjugated polymers, and defects in their structures can significantly hamper their photophysical, charge transport and stabilization properties [Zijie Qiu, Brenton A. G. Hammer, Klaus Müllen, Progress in Polymer Science 100 (2020) 101179]. For example, the synthesis of conjugated polymers can be complex and require precise control over molecular weight, polydispersity, and chemical structure to achieve desired optical and electronic properties. This complexity can limit scalability and increase production costs. Conjugated polymers always experience aggregation-caused quenching issues. Conjugated polymer has multiple absorptions due to its D-I-A complicated structure, which could lead to issues such as spillover or crosstalk in multicolor panel experiments.

Fluorescent non-conjugated polymer nanoparticles are not as well known. Research only focused on how to load more single-color dyes to get brighter polymer dyes with less aggregation induced quenching. [Otto S. Wolfbeis, Chem. Soc. Rev., 2015, 44, 4743-4768]. However, there isn't any research on Fluorescent non-conjugated polymeric tandem dyes. [Judit Morla-Folch, Guillem Vargas-Nadal, Tinghan Zhao, Cristina Sissa, Antonio Ardizzone, Siarhei Kurhuzenkau, Mariana Köber, Mehrun Uddin, Anna Painelli, Jaume Veciana, Kevin D. Belfield, and Nora Ventosa, ACS Applied Materials & Interfaces 2020, 12, 18, 20253-20262].

It is possible to get efficient Förster resonant energy transfer (FRET) in nonconjugated polymer if emission spectrum of the grafted donor groups overlaps well with the absorption of the grafted acceptor groups. This invention will open a path to get highly fluorescent non-conjugated polymeric tandem dyes.

Traditional non-conjugated polymers, which can be easily controlled over molecular weight, polydispersity, and chemical structure to achieve expected biocompatible, biodegradable, optical and electronic properties, have been marked by significant advancements in their synthesis, characterization, processing, and application. Fluorescent non-conjugated polymeric tandem dyes based on the non-conjugated polymer have the potentiality to be an emerging class of polymers that combine the desirable optoelectronic properties of conjugated polymers with the superior synthetic methodologies and stability of traditional non-conjugated polymers.

However, some of these prior techniques form large and irregular size polymers, resulting in strong fluorescence background noise in biological applications. Some of these prior techniques are difficult to quantitatively calculate loading ratio of dyes, resulting in poor reproducible performance. Some of these prior techniques have a complicated synthetic process. Therefore, there is a need for a new type of fluorescent dye system with very high brightness and good reproducible performance.

### BRIEF SUMMARY

Embodiments generally pertain to fluorescent non-conjugated polymeric tandem dyes, which can form highly fluorescent polymer nanoparticles or clusters, in which efficient Förster resonant energy transfer (FRET) between fluorescent donor groups and fluorescent acceptor groups give bright fluorescence. Some embodiments also provide design considerations in synthesizing these fluorescent non-conjugated polymeric tandem dyes, preparation methods for forming the polymer nanoparticles or clusters, and biological applications. Fluorescent nonconjugated polymeric tandem dyes bring forward unique properties of highly fluorescent polymer nanoparticle bioconjugates for clinical and biological applications, especially for flow cytometry application.

The present invention relates generally to fluorescent nanomaterials and, more particularly, to fluorescent non-conjugated polymer tandem dye nanoparticles useful for flow cytometry, biological detection, imaging, and related analytical applications. The disclosed nanoparticles comprise a non-conjugated polymer backbone formed from polymer backbone units connected predominantly through single bonds, including but not limited to carbon-carbon, carbon-hydrogen, carbon-oxygen, carbon-nitrogen, carbon-sulfur, carbon-silicon, silicon-oxygen, and phosphorus-oxygen bonds, thereby avoiding extended w-conjugation along the polymer backbone.

In accordance with various embodiments, the non-conjugated polymer backbone includes a plurality of pendant side chains covalently bonded to different backbone units. These pendant side chains may include one or more fluorescent donor monomers, one or more fluorescent acceptor dyes, one or more Förster resonance energy transfer (FRET) monomers, and one or more functional monomers bearing reactive groups. The spatial proximity of the donor monomers, optional FRET monomers, and acceptor dyes within the polymer nanoparticle enables efficient intraparticle energy transfer upon excitation of the fluorescent donor monomers at one or more defined excitation wavelengths.

The fluorescent donor monomers may comprise aromatic, heteroaryl, planar, or cyclic fluorescent compounds capable of absorbing excitation light and transferring energy through one or more FRET pathways. Representative donor fluorophores include, but are not limited to, acridine, coumarin, rhodamine, fluorescein, BODIPY, pyrene, quinoline, benzoxazole, anthracene, thiazole, benzothiazole, cyanine, diketopyrrolopyrrole (DPP), carbazole, indole, fluorene, squaraine, phthalocyanine, lanthanide complexes, and derivatives thereof, with absorption wavelengths spanning from the ultraviolet to near-infrared region. In certain embodiments, the fluorescent donor monomers include hydrophilic, hydrophobic, amphiphilic, or branched side chains to promote nanoparticle stability and solubility in aqueous environments.

In some embodiments, one or more FRET monomers are incorporated as pendant side chains on the non-conjugated polymer backbone to provide intermediate energy transfer steps between donor monomers and acceptor dyes. The FRET monomers may exhibit Stokes shifts ranging from a few nanometers to several hundred nanometers and may absorb and emit across a broad spectral range. The donor monomers, FRET monomers, and acceptor dyes may be incorporated into the polymer backbone through copolymerization with reactive monomers or may be post-grafted onto a pre-formed polymer backbone through reactions with complementary reactive functional groups.

The fluorescent acceptor dyes are selected to provide narrow emission bandwidths, high quantum yields, and emission spectra suitable for multiplexed optical detection, including full-spectrum flow cytometry. In some embodiments, the fluorescent acceptor dyes exhibit a full width at half maximum (FWHM) of less than about 65 nanometers. The acceptor dyes may comprise organic fluorescent dyes or metal-complex dyes and may be covalently attached to the polymer backbone or to the surface of polymer nanoparticles via post-grafting reactions, including click chemistry reactions.

The non-conjugated polymer backbone may be synthesized using a wide range of polymerization techniques, including free radical polymerization, anionic or cationic polymerization, living or controlled polymerization methods such as ATRP, RAFT, and NMP, ring-opening polymerization, polycondensation, polyaddition, coordination polymerization, and ring-opening metathesis polymerization. In certain embodiments, the polymer backbone is first synthesized with reactive functional groups, followed by post-grafting of fluorescent donor monomers, FRET monomers, acceptor dyes, and functional monomers through reactions involving amino, thiol, azide, alkyne, maleimide, carboxyl, hydroxyl, epoxide, acrylate, sulfonate, ester, or other reactive functional organic groups. Reactive functional organic groups, as exemplified above, are specific atoms or groups of atoms in organic molecules that dictate their chemical behavior, acting as the main sites for reactions such as additions, substitutions, and eliminations.

The pendant functional monomers may include mono-functional, di-functional, or multi-functional linkers or oligomers that enable subsequent bioconjugation of the nanoparticles to biomolecules such as antibodies, proteins, nucleic acids, or carbohydrates. These functional monomers may include linker backbones comprising alkyl chains, peptide chains, or polyethylene oxide chains with variable lengths ranging from a few repeating units to several thousand repeating units. In certain embodiments, the functional monomers are configured to enable sequential or simultaneous reactions with bioactive molecules and the polymer backbone.

The present invention further provides methods for preparing the fluorescent nonconjugated polymer tandem dye nanoparticles. In representative methods, a fluorescent nonconjugated polymer is dissolved in an organic solvent and then introduced into water under agitation or sonication, resulting in the formation of polymer nanoparticles through solvent removal and polymer entanglement. Amphiphilic surfactants may be added to stabilize and disperse the nanoparticles in aqueous solution. Fluorescent acceptor dyes and functional molecules may be subsequently coupled to the nanoparticles through post-grafting reactions, including click chemistry reactions.

In some embodiments, the invention also encompasses aqueous solutions containing the fluorescent non-conjugated polymer tandem dye nanoparticles dispersed in water with surfactants, as well as compositions in which the nanoparticles are bio-conjugated to antibodies or other bioactive molecules for use in biological assays, diagnostics, and flow cytometry.

Additionally, the invention includes related methods for producing fluorescent nanoparticle complexes based on inorganic nanoparticles, such as metal oxide nanoparticles, that are functionalized with reactive coupling agents, fluorescent dyes, and linker molecules, followed by direct bioconjugation to antibodies or other bioactive molecules.

Collectively, the fluorescent non-conjugated polymer tandem dye nanoparticles of the present invention provide tunable excitation and emission properties, narrow emission bandwidths, high brightness, and robust bioconjugation capabilities, making them particularly well suited for advanced multiplexed optical detection and biological analysis applications.

The embodiments are also generally summarized by the claims that follow below.

### BRIEF DESCRIPTIONS OF THE DRAWINGS

This patent or application file contains at least one drawing executed in color. Copies of this patent or patent application publication with color drawing(s) will be provided by the United States Patent and Trademark Office upon request and payment of the necessary fee.

The drawings, described below, are for illustration purposes only. The drawings are not intended to limit the scope of the embodiments in any way. These drawings and other features and embodiments are better understood with regards to the following description, appended claims, and accompanying drawings where:

FIG. 1 depicts a schematic structure of a fluorescent non-conjugated polymeric tandem dye.

FIG. 2 shows a process of the formation of nanoparticles from a non-conjugated polymer, a mechanism of FRET in nanoparticles to give fluorescence, and a bioconjugation with an antibody.

FIG. 3 shows examples of non-conjugated polymers with reactive groups.

FIGS. 4A-4B show examples of fluorescent donor monomers.

FIG. 5 shows examples of FRET monomers.

FIG. 6 shows examples of fluorescent acceptor dyes.

FIG. 7A-7C shows examples of functional groups (functional monomers).

FIG. 8 illustrates a schematic structure of a fluorescent non-conjugated polymeric tandem dye.

FIG. 9 illustrates an example process of forming fluorescent non-conjugated polymer tandem dye nanoparticles based on the non-conjugated polymer shown in FIG. 8.

FIG. 10 illustrates a schematic structure of a fluorescent non-conjugated polymeric dye.

FIG. 11 illustrates an example process of forming fluorescent non-conjugated polymer tandem dye nanoparticles based on the non-conjugated polymer shown in FIG. 10.

FIG. 12 illustrates an example process of forming a deep ultra-violet excitable fluorescent non-conjugated polymer.

FIG. 13 illustrates an example process of forming fluorescent non-conjugated polymer nanoparticles based on the non-conjugated polymer formed by the process shown in FIG. 12.

FIG. 14A illustrates a dot plot of density plotting side scatter (SSC) versus a deep ultra-violet detection channel (DUV3) running whole blood cells stained with CD4-320ex360 at 1000 ng through a flow cytometer.

FIG. 14B illustrates a chart plotting absorption and fluorescence spectra of CD4-320ex360 conjugate in a phosphate-buffered saline (PBS) buffer associated with the dot plot shown in FIG. 14A.

FIG. 15A illustrates a dot plot of density plotting side scatter (SSC) versus a deep ultra-violet detection channel (DUV3) after running whole blood cells stained with CD4-320ex372 at 1000 ng through a flow cytometer.

FIG. 15B illustrates a chart plotting absorption and fluorescence spectra of CD4-320ex372 conjugate in a PBS buffer associated with the dot plot shown in FIG. 15A.

FIG. 16A illustrates a dot plot of density plotting side scatter (SSC) versus a deep ultra-violet detection channel (DUV3) after running whole blood cells stained with CD4-320ex385 at 1000 ng through a flow cytometer.

FIG. 16B illustrates a chart plotting absorption and fluorescence spectra of CD4-320ex385 conjugate in a PBS buffer associated with the dot plot shown in FIG. 16A.

FIG. 17A illustrates a dot plot of density plotting side scatter (SSC) versus a deep ultra-violet detection channel (DUV4) after running whole blood cells stained with CD4-320ex435 at 500 ng through a flow cytometer.

FIG. 17B illustrates a chart plotting absorption and fluorescence spectra of CD4-320ex435 conjugate in a PBS buffer associated with the dot plot shown in FIG. 17A.

FIG. 18A illustrates a dot plot of density plotting side scatter (SSC) versus a deep ultra-violet detection channel (DUV5) after running whole blood cells stained with CD4-320ex457 at 500 ng through a flow cytometer.

FIG. 18B illustrates a chart plotting absorption and fluorescence spectra of CD4-320ex457 conjugate in a PBS buffer associated with the dot plot shown in FIG. 18A.

FIG. 19 is a perspective view of one or more vials of bioconjugated nanoparticles and instructions in a reagent kit.

FIG. 20 is a basic conceptual diagram of a flow cytometer system.

FIG. 21 is a conceptual diagram of a fluorochrome, an antibody, and a cell.

FIG. 22 is a conceptual diagram of forming a reference sample with a bead.

FIG. 23A is an overall method for performing an experiment with a biological sample and/or running calibration beads through a flow cytometer.

FIG. 23B is a diagram of a calibrating process of a flow cytometer with single stained compensation controls to generate an initial spillover matrix or reference matrix with levels of compensation.

FIG. 23C is a diagram of running a sample through the flow cytometer resulting in a mixed sample event vector with an overlapping spectral profile due to multi-stained cells or particles.

FIG. 23D is a diagram of a processing using an inverse matrix (determined from the initial spillover matrix and/or the initial reference matrix with fine adjustments) on the event data to generate a compensated sample event vector or an unmixed sample event vector.

FIG. 24 (24-1 and 24-2) is a schematic diagram of a full spectrum flow cytometer.

FIG. 25 shows configuration details of the photo detectors in the detector modules for a full spectrum flow cytometer.

FIG. 26 (26-1 and 26-2) illustrates the individual spectrum signature of each color laser and combined full spectrum signature of an exemplary fluorochrome.

### DETAILED DESCRIPTION

In the following detailed description of the disclosed embodiments, numerous specific details are set forth in order to provide a thorough understanding. However, it will be obvious to one skilled in the art that the disclosed embodiments can be practiced without these specific details. In other instances, well known methods, procedures, components, and subsystems have not been described in detail so as not to unnecessarily obscure aspects of the disclosed embodiments.

Polymers and monomers are described herein. A polymer is a large molecule, or macromolecule, composed of small repeating singular molecular structural units called monomers. The repeating molecular units are joined together chemically through covalent bonds. The disclosed embodiments oftentimes refer to fluorescent donor monomers and FRET monomers. It should be considered that the scope of the embodiments also encompasses repeating the monomer units. Thus, embodiments of the invention can also encompass fluorescent donor polymers and FRET polymers, etc. Fluorescent donor monomers and donor polymers can thus be interchangeably referred to as donor units, e.g. pendant fluorescent donor units or pendant functional units.

The disclosed embodiments include methods, apparatus, systems, and compositions of matter for nanoparticle fluorescent dye complexes. The nanoparticle fluorescent dye complex has a very high brightness, and its synthesis is disclosed herein. Disclosed embodiments can also include inorganic nanoparticle fluorescent dye complexes.

Generally, a nanoparticle (NP) is a small particle that ranges in size between 1 nanometer (nm) to 100 nanometers (nm) in size. Undetectable by the human eye, nanoparticles can exhibit significantly different physical and chemical properties to their larger material counterparts. Material properties change as the size of the material object approaches the atomic scale. This is due to the surface area to volume ratio increasing, resulting in the material's surface atoms dominating the material performance. Owing to their very small size, nanoparticles have a very large surface area to volume ratio when compared to bulk material, such as powders, plate and sheet. This feature enables nanoparticles to possess unexpected optical, physical and chemical properties, as they are small enough to confine their electrons and produce quantum effects.

Nanoparticles can be formed from organic or inorganic material. Nanoparticles formed from inorganic material can be referred to inorganic nanoparticles. Nano-sized inorganic particles (inorganic nanoparticles) of either simple or complex nature can display unique, physical and chemical properties. Inorganic nanoparticles represent an increasingly important material in the development of novel nanodevices which can be used in numerous physical, biological, biomedical and pharmaceutical applications.

Conjugation can refer to the overlap of one p-orbital with another across an adjacent σ bond. To be considered conjugated, two or more pi bonds must be separated by only one single bond Alternating single and double bonds create a conjugated pi bond system across multiple atoms that lowers the energy and stabilizes the molecule or ion. The backbone of a conjugated polymer has alternating σ (sigma) and π (pi) bonds.

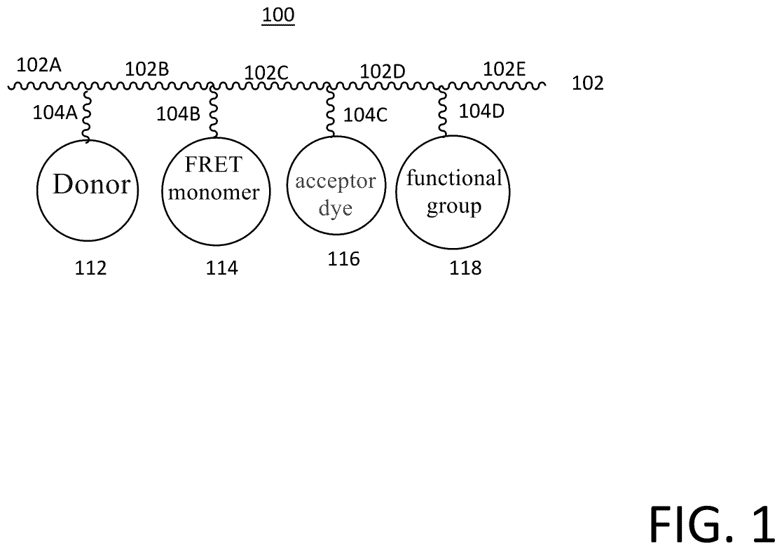

In FIG. 1, non-conjugated polymer backbone units 102A-102E are single bond units mainly built by carbon-carbon single bonds (C—C), and/or carbon-hydrogen single bonds (C—H), and/or carbon-sulfur single bonds (C—S), and/or carbon-oxygen single bonds (C—O), and/or carbon-nitrogen single bonds (C—N), and/or carbon-silicon single bonds (C—Si), and/or silicon-oxygen single bonds (Si—O), and/or phosphorus-oxygen single bonds (P—O), or a combination of the above single bonds.

The non-conjugated polymer backbone 102 can be made by copolymerizing reactive monomers which connect with fluorescent donor monomers, FRET monomers, functional groups (functional monomers), respectively. Copolymerization method can be free radical polymerization, anionic polymerization, cationic polymerization, polycondensation, polyaddition, coordination polymerization, ring-opening polymerization, atom transfer radical polymerization (ATRP), reversible addition fragmentation chain transfer (RAFT), nitroxide mediated polymerization (NMP), ring-opening metathesis polymerization (ROMP), etc.

Electrons in conjugated π systems are shared by all adjacent sp2- and sp-hybridized atoms that contribute overlapping, parallel p atomic orbitals. As such, the atoms and π-electrons involved behave as one large bonded system. In non-conjugates polymers electrons do not freely travel along the non-conjugated polymer backbone. Energy transfer occurs in conjugated systems by intrachain energy transfer and Förster resonant energy transfer (FRET). Fluorescent nonconjugated polymers transfer energy by FRET.

Förster resonant energy transfer (FRET), sometimes referred to as fluorescence resonance energy transfer, is a nonradiative energy transfer between two light sensitive molecules or chromophores. “Förster resonance energy transfer” is preferred over “fluorescence resonance energy transfer” because energy is not actually transferred by fluorescence. A donor chromophore, initially in its electronic excited state, may transfer energy to an acceptor chromophore through nonradiative dipole-dipole coupling.

The theory supporting energy transfer is based on the concept of treating an excited fluorophore as an oscillating dipole that can undergo an energy exchange with a second dipole having a similar resonance frequency. In this regard, resonance energy transfer is analogous to the behavior of coupled oscillators, such as a pair of tuning forks vibrating at the same frequency. In contrast, radiative energy transfer requires emission and reabsorption of a photon and depends on the physical dimensions and optical properties of the specimen, as well as the geometry of the container and the wavefront pathways. The fluorescent donor monomers have a light receiving property and can be grafted on a non-conjugated polymer backbone. Alternatively, the fluorescent donor monomers could have reactive groups, which can be copolymerized with other monomers to form a non-conjugated polymer, and pendant fluorescent donor monomers as a side chain. Reactive groups can also be post-grafted on non-conjugated polymer backbone by reacting with reactive groups on a non-conjugated polymer backbone.

Reactive groups on fluorescent donor monomers include but not limited to amino, thiol, alkyl, halide, azides, sulfonic acid, alkyne, phenol, aldehyde, maleimide, hydroxyl, nitrile, carboxylic acid, or its ester, or its salts, acetal, isocyanate, epoxide, acrylate, alkyl halides, aryl halides, sulfonate (tosyl, mesyl), boric acid or its ester, nitrophenyl carbonate, biotins, folic acid, methacrylate, mercapto, tetrafluorophenyl esters, succinimidyl ester, pentafluorophenyl ester, hydrazides, vinyl sulfone, dibenzocyclooctyne group (DBCO), methyltetrazine and other functional organic groups.

A tandem dye is a type of fluorescent dye used in biological and biomedical research, particularly in flow cytometry, fluorescence microscopy, and other fluorescence-based techniques. It consists of two fluorescent molecules (fluorophores or dyes). Usually, a donor dye and an acceptor dye are covalently bonded to each other for energy transfer between them. In the disclosed embodiments, the fluorescent donor monomer and the fluorescent acceptor dye are not covalently bonded to each other. However, the energy transfer between the fluorescent donor monomer and the fluorescent acceptor dye still occurs due to the Förster Resonance Energy Transfer (FRET) process, where the fluorescent donor monomer absorbs light and transfers energy to the fluorescent acceptor dye. The fluorescent donor monomers have a light harvesting function which provides a large absorption at specific laser excitations of a center wavelength. Typically, the fluorescent acceptor dye emits light (emission wavelength) at a longer wavelength than light is absorbed (absorption wavelength) by the fluorescent donor monomer.

Referring now to FIG. 1, a schematic structure of a fluorescent non-conjugated polymeric tandem dye 100 is shown. In some embodiments, the fluorescent non-conjugated polymeric tandem dye 100 comprises a non-conjugated polymer backbone 102, and one or more fluorescent donor monomers 112, one or more FRET monomers 114, one or more fluorescent acceptor dyes 116, and one or more functional groups (functional monomers) 118. The fluorescent non-conjugated polymeric tandem dye 100 is a tandem dye given that it includes both the fluorescent donor monomers 112 and the fluorescent acceptor dyes 116.

The fluorescent donor monomers 112 can be grafted onto the non-conjugated polymer backbone 102 as side chains referred to as a pendant fluorescent donor monomer. The FRET monomers 114 can be grafted onto the non-conjugated polymer backbone 102 as side chains referred to as pendant FRET monomers. The fluorescent acceptor dyes 116 can be grafted onto the non-conjugated polymer backbone as side chains referred to as pendant fluorescent acceptor dyes. The functional groups (functional monomers) 118 can be grafted onto the non-conjugated polymer backbone as side chains referred to as pendant functional groups (pendant functional monomers). These side chains are randomly bonded along the backbone 102 in any order. While a single grouping of side chains are shown in FIG. 1, there can be one or more of each side chain randomly coupled to the backbone 102 to provide one or more fluorescent donor monomers 112, one or more FRET monomers 114, one or more of fluorescent acceptor dyes 116, and one or more of functional groups (functional monomers) 118.

The fluorescent donor monomer 112, the FRET monomer 114, the fluorescent acceptor dye 116, and the function group 218 can be attached to the non-conjugated polymer backbone 102 by a single bond, generally a sigma bond. The non-conjugated polymer backbone 102 can comprise backbone subsections 102A-102E. Each backbone subsection is linked to subsequent and/or preceding backbone subsections by sigma bonds or single bonds.

In contrast to conjugated polymers, non-conjugated polymeric embodiments lack alternating σ (sigma) and π (pi) bonds. A non-conjugated polymer backbone does not have alternating single and multi-bonds in their backbone chain. A non-conjugated polymer backbone is generally built by carbon-carbon single bonds (C—C), carbon-hydrogen single bonds (C—H), carbon-sulfur Single bonds (C—S), or carbon-oxygen single bonds (C—O), or carbon-nitrogen single bonds (C—N), or carbon-silicon single bonds (C—Si), silicon-oxygen single bonds (Si—O), phosphorus-oxygen single bonds (P—O), or combination of above single bonds. The nonconjugated polymer backbone 102 can comprise one of the aforementioned single bonds as backbone subcomponents 102A-102E. Single bonds are generally rigid in the sense that they do not have pi electrons. Therefore, a non-conjugated polymer backbone comprising only single bonds, will not be conductive along the polymer backbone.

In some embodiments, non-conjugated polymers backbone can be linear polymers, branched polymers, star polymers, dendritic polymers, crosslinked polymers, and networked polymers, etc.

In some embodiments, non-conjugated polymers can be made by copolymerizing fluorescent donor monomers 112, and/or FRET monomers 114, and/or fluorescent acceptor dyes 116, and/or functional groups 118, respectively. Copolymerization method can be free radical polymerization, anionic polymerization, cationic polymerization, living anionic polymerization, polycondensation, polyaddition, coordination polymerization, ring-opening polymerization, atom transfer radical polymerization (ATRP), reversible addition fragmentation chain transfer (RAFT), nitroxide mediated polymerization (NMP), ring-opening metathesis polymerization (ROMP), etc.

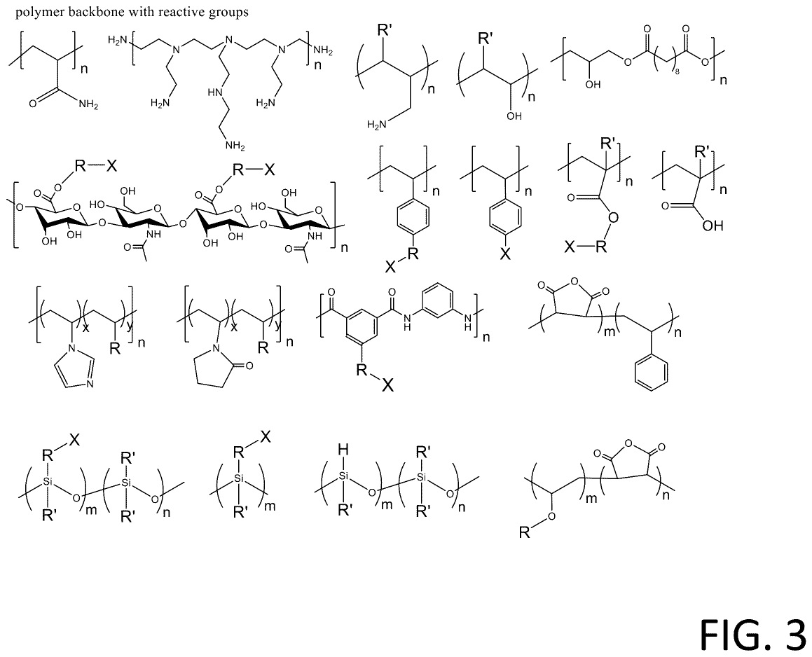

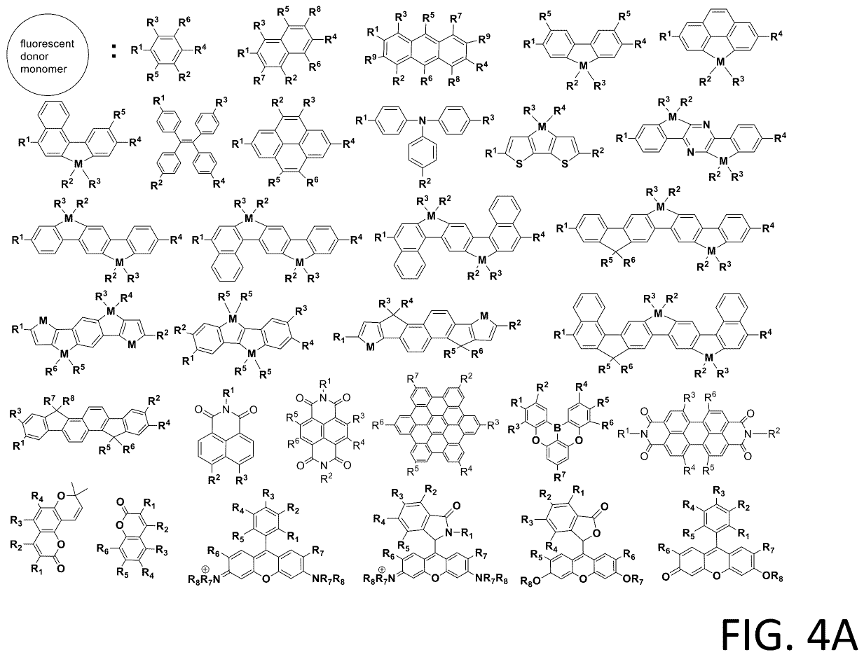

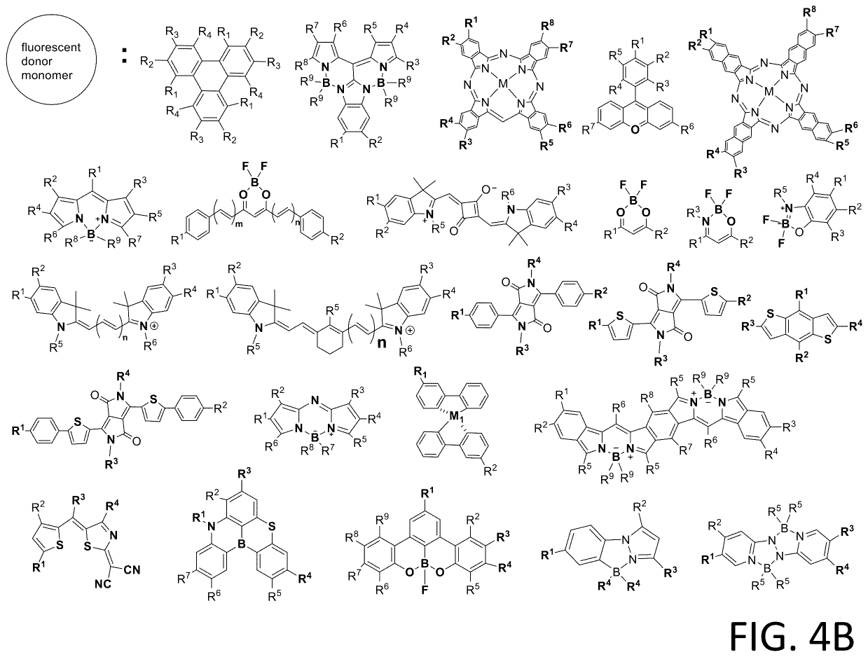

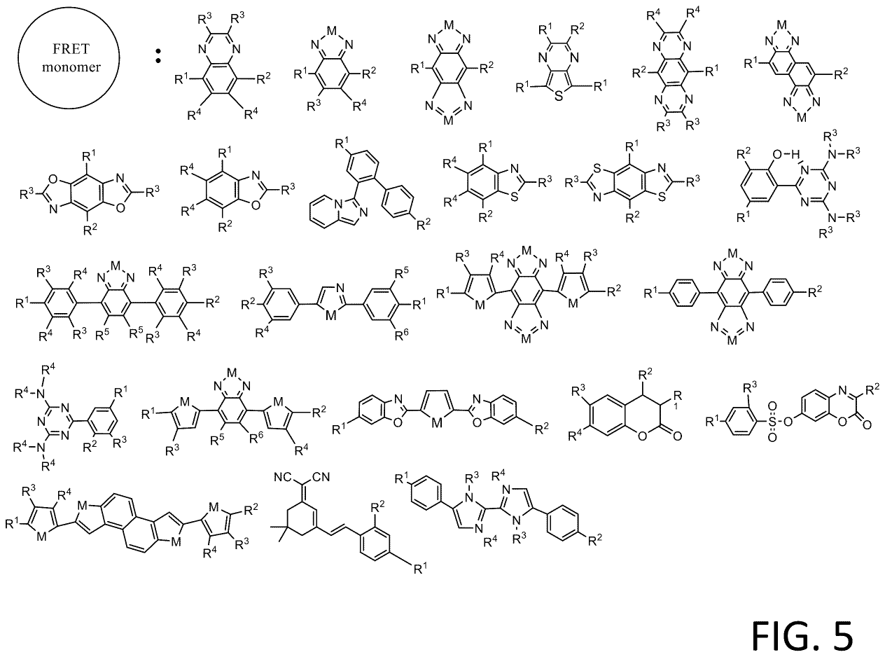

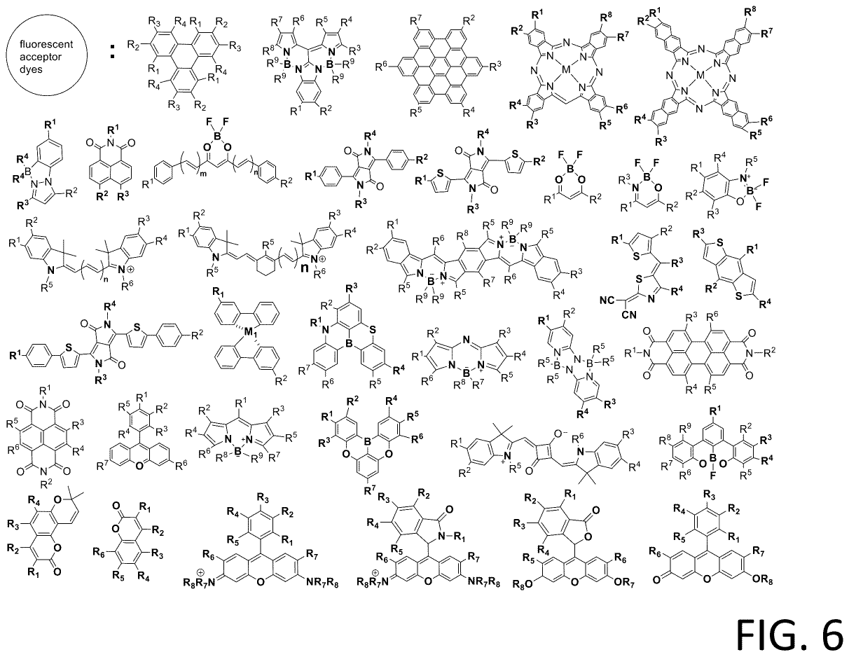

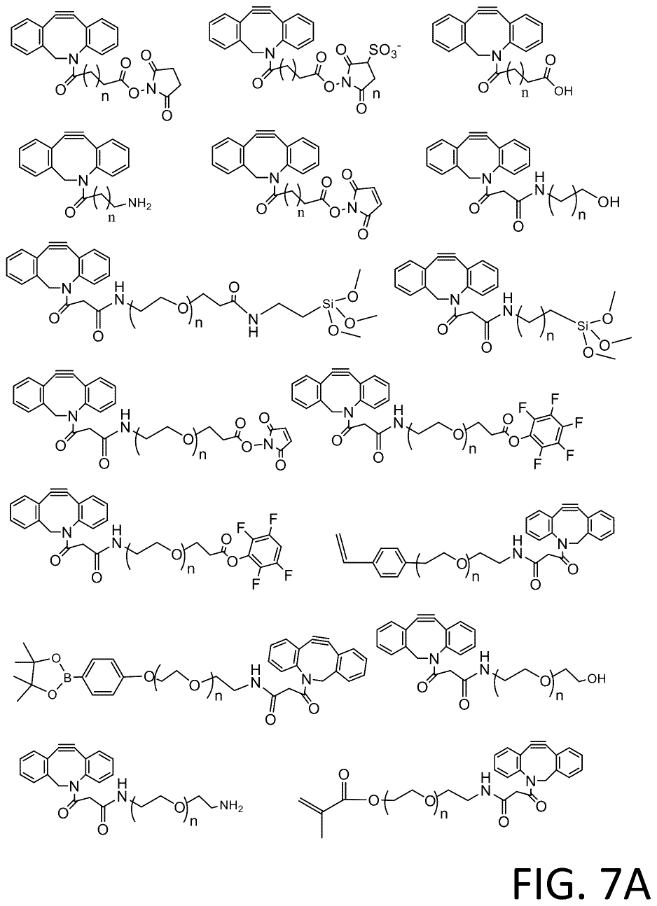

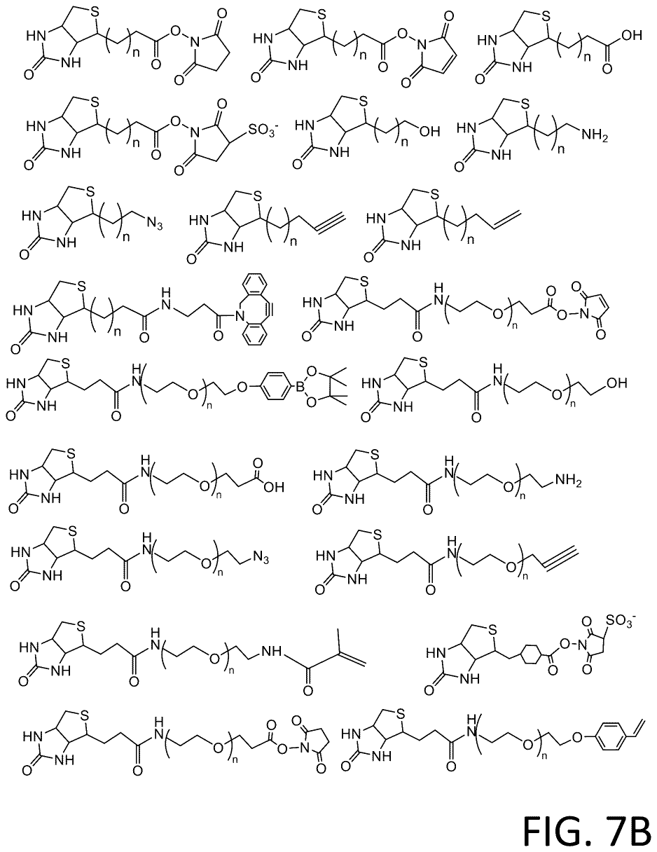

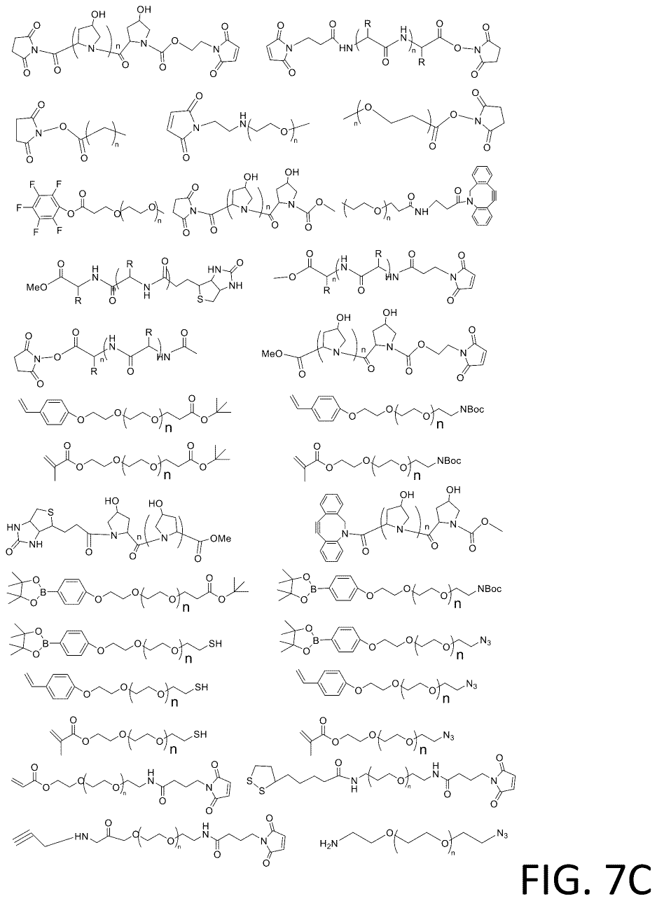

In some embodiments, a non-conjugated polymer backbone 102 can be synthesized first with attached reactive groups. Examples of reactive groups can be found in FIG. 3 attached to a non-conjugated polymer. Fluorescent donor monomers 112, and/or FRET monomers 114, and/or fluorescent acceptor dyes 116, and/or functional groups (functional monomers) 118 can then be grafted onto the non-conjugated polymer backbone 102 by reacting with reactive groups on polymer backbone. Examples of fluorescent donor monomers can be found in FIGS. 4A and 4B. Examples of FRET monomers 2014 can be found in FIG. 5. Examples of fluorescent acceptor dyes 116 can be found in FIG. 6. Examples of functional groups (functional monomers) can be found in FIGS. 7A, 7B and 7C.

Disclosed embodiments comprise a fluorescent non-conjugated nanoparticle. The fluorescent non-conjugated nanoparticle further comprises a polymer backbone that is hydrophobic and non-conjugated. When injecting nanoparticles into water, the hydrophobic polymer backbone can form nanoparticles. The nanoparticles in water can form spherical structures. A surfactant can be used to control the nanoparticle size and deter entanglement of nanoparticles.

Disclosed embodiments can be packaged into a resealable container as a solution including water; a plurality of fluorescent non-conjugated polymer tandem dye nanoparticles as recited in claim 1 in the water; and an amphiphilic surfactant in the water to disperse the plurality of fluorescent non-conjugated polymer tandem dye nanoparticles in the water. The solution can further include a plurality of antibodies respectively bio-conjugated to the plurality of fluorescent non-conjugated polymer tandem dye nanoparticles. Each of the plurality of fluorescent nonconjugated polymer tandem dye nanoparticles can further include one or more pendant FRET monomers each bonded to a different non-conjugated polymer backbone unit of the nonconjugated polymer backbone.

In some embodiments the fluorescent polymer nanoparticles can be prepared as follows. Polymer is dissolved in methanol, then polymer solution is injected into water under sonication. Polymer nanoparticles solution is concentrated using centrifuge. Finally, fluorescent polymer nanoparticles aqueous solution is obtained with certain concentration. Concentrated fluorescent polymer nanoparticles can further react with NHS-dye to get fluorescent polymer tandem dye.

Traditionally, a water-soluble polymer was desired for biological use. Water soluble polymers, while biologically useful, can have disadvantages. For instance, the polymer chain is very long and if it is not water soluble it tends to self-aggregate and cause interchain interaction. A single linear chain polymer that aggregates can cause issues with the fluorochrome emission.

An advantage of the spherical structure of the hydrophobic nanoparticles is a reduction or elimination of single chain aggregation. After formation of the spherical shaped nanoparticle, it can be advantageous to make the spherical shaped nanoparticle water soluble to disperse in water to avoid nanoparticle to nanoparticle aggregation. By removing the solvent from the mixture to allow the fluorescent non-conjugated polymer to entangle in the presence of water, thus forming water-soluble fluorescent non-conjugated polymer tandem dye nanoparticles.

A water-soluble backbone with attached fluorescent donor monomer, FRET monomer, acceptor dye, and functional group can disperse in the water instead of entangling together. In contrast, the hydrophobic backbone of embodiments of the invention can entangle with other nanoparticles advantageously leading to higher absorption intensity due to a plurality of polymer chains entangling together. However, it can also be advantageous to form the nanoparticle ball or spheres and then add a surfactant or other chemical to cause the nanoparticle to disperse, thereby limiting aggregation with other nanoparticles. The surfactant can be an amphiphilic surfactant with both hydrophilic and hydrophobic properties.

Several novel embodiments pertain to enhanced energy transfer. In some embodiments, the structure of the nanoparticle allows for more orderly energy transfer with less interchain interaction. Additionally, in some embodiments, a FRET monomer can be added to the non-conjugated polymeric nanoparticle backbone to enhance energy transfer by facilitating more efficient energy transfer by FRET. Embodiments of the invention evidence higher absorption of excitation energy by the fluorescent donor monomer and more efficient FRET transfer to the acceptor. The fluorescence spectra of the various acceptor dyes evidence clearly defined peaks at the longer wavelengths.

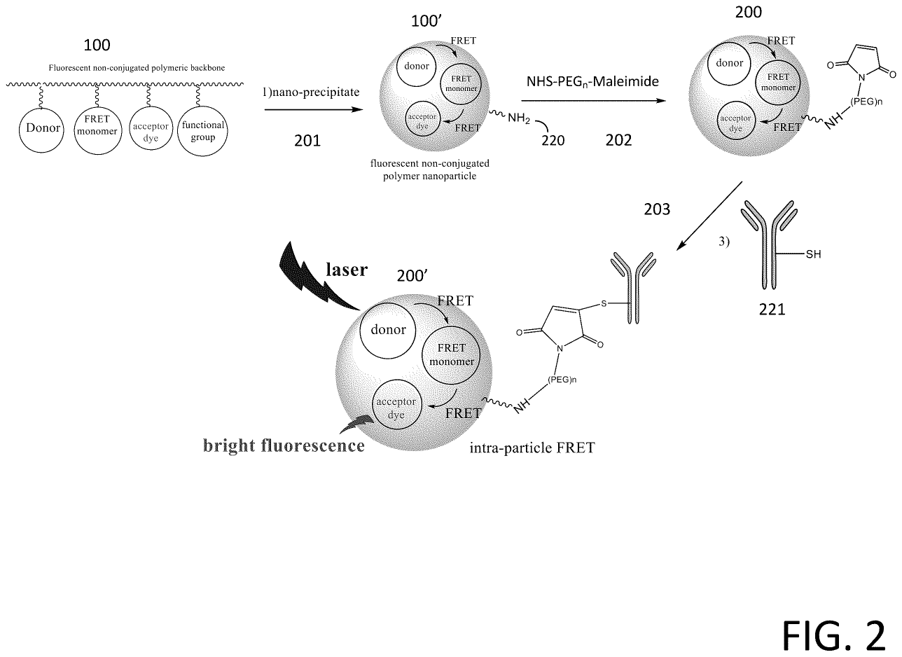

Referring now to FIG. 2, a process for the formation of a fluorescent nonconjugated polymeric tandem dye nanoparticle 200′ is shown. Generally, the process includes a formation of nanoparticles from a non-conjugated polymer, a bioconjugation with an antibody, and using a mechanism of FRET in nanoparticles to give fluorescence.

In step 201, the fluorescent non-conjugated polymer 100 can be injected into water to nano-precipitate and form the fluorescent non-conjugated polymer nanoparticles 100′. In some embodiments, the fluorescent non-conjugated polymer 100, dissolved in solution, is injected into water under sonification to nano-precipitate and form the fluorescent nonconjugated polymer nanoparticles 100′ in a solution. The fluorescent non-conjugated polymer nanoparticles 100′ can form a structural sphere with reactive groups 220 on the surface of the sphere. The concentration of the fluorescent non-conjugated polymer nanoparticles 100′ in solution can be further concentrated by evaporation or centrifuge.

In this example, the reactive group 220 is NH₂ or a primary amine. NH₂ is a reactive group on the end of the functional group 118 attached to the polymeric backbone. Alternatively reactive group 220 can be attached to the polymer backbone 102 during synthesis.

Reactive groups 220 on the non-conjugated polymer backbone include but are not limited to amino, thiol, alkyl, halide, Azides, Sulfonic Acid, alkyne, Phenol, aldehyde, maleimide, hydroxyl, Nitrile, Carboxylic acid, or its ester, or its salts, acetal, isocyanate, epoxide, acrylate, alkyl halides, aryl halides, sulfonate (tosyl, mesyl), nitrophenyl carbonate, Biotins, folic acid, methacrylate, mercapto, tetrafluorophenyl esters, succinimidyl ester, pentafluorophenyl ester, hydrazides, vinyl sulfone, dibenzocyclooctyne group (DBCO), methyltetrazine and other functional organic groups.

In step 202, the concentrated fluorescent non-conjugated polymeric nanoparticles 100′ can further react with NHS-PEGn-Maleimide in a PEGylation process to produce a fluorescent non-conjugated polymeric tandem dye nanoparticle 200.

In step 203, the fluorescent non-conjugated polymeric tandem dye nanoparticle 200 is bioconjugated with an antibody 221 to create a fluorescent non-conjugated polymeric tandem dye nanoparticle 200′. The functional group 118 is used to connect the fluorescent nonconjugated polymer tandem dye nanoparticle 200 onto the antibody 221 via the bioconjugation reaction. The functional group 118 stretches out to surface of nanoparticle and further reacts with antibody via the bioconjugation reaction. The functional group 118 is still attached to the backbone 102 when the fluorescent non-conjugated polymer tandem dye nanoparticle 200′ is fully formed. Examples of functional groups 118 are shown in FIGS. 7A-7C.

While one fluorescent donor monomer, one FRET monomer, and one fluorescent acceptor dye are shown being a part of fluorescent non-conjugated polymer tandem dye nanoparticle 200′, there can be a plurality of each. The merit of non-conjugated polymer nanoparticles is to incorporate more fluorescent donor monomers into the polymer which can highly increase the absorption intensity of the polymer. Brightness of dye is related to extinction coefficient (ξ) of the dye and the quantum yield (Φ) of the dye. Brightness of dye is equal to extinction coefficient (ξ) of dye times the quantum yield (Φ) of the dye, where the extinction coefficient (ξ) is proportional to absorption intensity of donor dyes. This is one reason why a fluorescent non-conjugated polymer tandem dye nanoparticle can provide a greater level of brightness.

The fluorescent donor monomer 112 of the fluorescent non-conjugated polymeric tandem dye nanoparticle 200′ can be excited by a laser beam of light from a laser causing an energy transfer by a first Förster resonant energy transfer (FRET) process from the fluorescent donor monomer 112 to a FRET monomer 114 within the particle and subsequently an energy transfer from the FRET monomer 114 to the pendant acceptor dye 116 by a second FRET processes. The FRET monomer provides an inter particle FRET that provides a more efficient intra-particle energy transfer process leading to a brighter fluorescence of the pendent acceptor dye 116.

The fluorescent donor monomers typically contain one or several combined aromatic compounds, heteroaryl compounds, or combination of aromatic and heteroaryl compounds, or planar or cyclic molecules with several bonds. The fluorescent donor monomers further comprise fluorescent donor dyes including but not limited to acridine-based monomers, coumarin-based monomers, rhodamino-based monomers, fluorescein-based monomers, BODIPY-based monomers, pyrene-based monomers, Quinoline-based monomers, benzoxazole-based monomers, anthracene-based monomers, thiazole-based monomers, benzothiazole-based monomers, cyanine-based monomers, diketopyrrolopyrrole (DPP)-based monomers, carbazole-based monomers, indole-based monomers, fluorene-based monomers, lanthanide-based monomers, squaraine-based monomers, and other organic fluorescent dyes with absorption between 260 nm and 1000 nm.

The fluorescent donor monomers should have side chains such as hydrophilic chains, or hydrophobic chains, or amphiphilic chains, or branched hydrophilic chains, or branched hydrophobic chains, or branched amphiphilic chains, etc.

In summary, a method for preparing fluorescent non-conjugated polymer nanoparticles includes providing a mixture comprising a solvent, an amphiphilic surfactant, and a fluorescent polymer dissolved into the solvent; and then removing said solvent from said mixture to allow said fluorescent polymer to entangle in the presence of said water, thus forming said water-soluble fluorescent polymer nanoparticles.

Referring now to FIG. 3, examples of non-conjugated polymers with reactive groups are shown. In some embodiments, the reactive groups “X” on non-conjugated polymer backbone side can be amino, thiol, alkyl, halide, Azides, Sulfonic Acid, alkyne, Phenol, aldehyde, maleimide, hydroxyl, Nitrile, Carboxylic acid, or its ester, or its salts, acetal, isocyanate, epoxide, acrylate, alkyl halides, aryl halides, sulfonate (tosyl, mesyl), boric acid or its ester, nitrophenyl carbonate, Biotins, folic acid, methacrylate, mercapto, tetrafluorophenyl esters, succinimidyl ester, pentafluorophenyl ester, hydrazides, vinyl sulfone, dibenzocyclooctyne group (DBCO), methyltetrazine and other functional organic groups.

The organic group “R” connecting reactive group X and polymer backbone include, but are not limited to, alkyl, substituted alkyl, alkoxy, substituted alkoxy, aryl, substituted aryl, heteroaryl, heterocycle, substituted heteroaryl, polyethylene glycol (PEG) oligomer chain, etc. side group “R′” on polymer backbone include, but are not limited to, hydrogen, alkyl, substituted alkyl, alkoxy, substituted alkoxy, aryl, substituted aryl, heteroaryl, heterocycle, substituted heteroaryl, polyethylene glycol (PEG) oligomer chain, etc.

In some embodiments, reactions between reactive groups on the non-conjugated polymer backbone 102 and reactive groups on fluorescent donor monomers 112, FRET monomers 114, fluorescent acceptor dyes 116, and functional groups 118 can be substitution reaction, addition reaction, elimination reaction, Redox reaction, condensation reaction, cycloaddition reaction, click chemistry reaction, photochemistry reaction, Suzuki coupling reaction, Stille coupling reaction, Sonogashira coupling reaction, Heck reaction, Mcmurray and Knoevenagel reaction, Wittig reaction, Horner reaction, etc.

In some embodiments, absorption peak of fluorescent donor monomers should fall into specific laser excitation wavelength, fluorescent donor monomers 112 typically contain one or several combined aromatic compounds, heteroaryl compounds, or planar or cyclic molecules with several bonds. The fluorescent donor monomers further comprise fluorescent donor dyes including but not limited to acridine or its derivatives, coumarin or its derivatives, rhodamine or its derivatives, fluorescein or its derivatives, BODIPY or its derivatives, pyrene or its derivatives, Quinoline or its derivatives, benzoxazole or its derivatives, anthracene or its derivatives, thiazole or its derivatives, benzothiazole or its derivatives, cyanine or its derivatives, diketopyrrolopyrrole (DPP) or its derivatives, carbazole or its derivatives, indole or its derivatives, fluorene or its derivatives, lanthanide or its derivatives, squaraine or its derivatives, and other organic fluorescent dyes with absorption between 260 nm and 1000 nm. Fluorescent donor monomers should have reactive groups, including but not limited to amino, thiol, alkyl, halide, Azides, Sulfonic Acid, alkyne, Phenol, aldehyde, maleimide, hydroxyl, Nitrile, Carboxylic acid, or its ester, or its salts, acetal, isocyanate, epoxide, acrylate, alkyl halides, aryl halides, sulfonate (tosyl, mesyl), boric acid or its ester, nitrophenyl carbonate, Biotins, folic acid, methacrylate, mercapto, tetrafluorophenyl esters, succinimidyl ester, pentafluorophenyl ester, hydrazides, vinyl sulfone, dibenzocyclooctyne group (DBCO), methyltetrazine and other functional organic groups.

In some embodiments, besides reactive groups on fluorescent donor monomers 112, mentioned above, which are already included in one of side groups (R1 to R9) on core of fluorescent donor monomer in FIGS. 4A, 4B, the other side groups (R1 to R9) can be independently selected from, but are not limited to, hydrogen, deuterium, halide, alkyl, substituted alkyl, alkoxy, substituted alkoxy, aryl, substituted aryl, heteroaryl, heterocycle, substituted heteroaryl, polyethylene glycol (PEG) oligomer chain, etc. R1 to R9 side groups may also be one or several fluorescent donor monomers or combined different fluorescent donor monomers connected with each other via covalent bond. “M” in FIG. 4A represents different atoms, including but not limited to carbon, silicon, sulfur, nitrogen, germanium, etc. The “M” in FIG. 4B can represent different atoms, including but not limited to zinc, magnesium, ferric, iron, cobalt, manganese, platinum, silicon, nickel, copper, aluminum, etc.

In some embodiments, alkyl and substituted alkyl side groups mentioned above, include but are not limited to, monovalent hydrocarbon chain with carbon atoms from 1 to 50. It can be linear chain or branched chain. Carbon atoms in the alkyl chain could also be optionally replaced with a heteroatom such as oxygen, nitrogen, sulfur, N—R, etc.

In other embodiments, alkoxy, substituted alkoxy side groups mentioned above refer to oxygen atom directly connecting to core of fluorescent donor monomer, include, but are not limited to, methoxy, ethoxy, n-propoxy, iso-propoxy, n-butoxy, t-butoxy, and the like, substituted alkoxy include, but are not limited to alkenyl-oxyl, substituted cycloalkyl-oxyl, substituted cycloalkenyl-oxyl, etc.

In some embodiments, aryl, substituted aryl side groups mentioned above refer to aromatic compounds or combined aromatic compounds, include, but are not limited to benzene, naphthalene, anthracene, pyrene, fluorene, carbazole, acenaphthylene, azulene, chrysene, coronene, fluoranthene, hexaphene, indane, indene, octacene, acephenanthrylene, octaphene, octalene, ovalene, pentacene, pentalene, pentaphene, perylene, phenalene, phenanthrene, picene, pleiadene, pyranthrene, triphenylene, trinaphthalene, triphenylamine, etc.

In some embodiments, polyethylene glycol (PEG) mentioned previously, refer to an oligomer made from repeating units of ethylene oxide, with the general formula H—(O—CH2—CH2)n—OH, where “n” can vary, indicating the number of ethylene oxide units.

In some embodiments, FRET monomers 114 should have a large stokes shift. The range of stokes shift ranges between 5 nanometers to 400 nanometers with its absorption peak ranging between 280 nm and 1100 nm. FRET monomers 114 typically contain one or several combined aromatic compounds, heteroaryl compounds, or planar or cyclic molecules with several bonds. FRET monomer can have reactive groups, including but not limited to amino, thiol, alkyl, halide, Azides, Sulfonic Acid, alkyne, Phenol, aldehyde, maleimide, hydroxyl, Nitrile, Carboxylic acid, or its ester, or its salts, acetal, isocyanate, epoxide, acrylate, alkyl halides, aryl halides, sulfonate (tosyl, mesyl), boric acid or its ester, nitrophenyl carbonate, Biotins, folic acid, methacrylate, mercapto, tetrafluorophenyl esters, succinimidyl ester, pentafluorophenyl ester, hydrazides, vinyl sulfone, dibenzocyclooctyne group (DBCO), methyltetrazine and other functional organic groups for example in FIG. 5.

In some embodiments, besides reactive groups on FRET monomers 114, mentioned previously, which are already included in one of side groups (R1 to R9) on core of FRET monomers 114 shown in FIG. 5, the other side groups (R1 to R9) can be independently selected from, but are not limited to, alkyl, substituted alkyl, alkoxy, substituted alkoxy, aryl, substituted aryl, heteroaryl, heterocycle, substituted heteroaryl, polyethylene glycol (PEG) oligomer chain, etc. R1 to R9 side groups may also be one or several FRET monomers connected with each other via covalent bond. The “M” used in FIG. 5 is a variable representing different atoms that can be used, including but not limited to carbon, oxygen, silicon, sulfur, nitrogen, selenium, germanium, etc.

Referring now to FIG. 6, examples of fluorescent acceptor dyes 116, include but are not limited to, acridine-based monomers, coumarin or its derivatives, rhodamine or its derivatives, fluorescein or its derivatives, organic BF2 complexes with N,N-bidentate, O,O-bidentate, N,O-bidentate, N,C-bidentate compounds its derivatives, pyrene or its derivatives, Quinoline or its derivatives, benzoxazole-based monomers, thiazole-based monomers, cyanine or its derivatives, diketopyrrolopyrrole (DPP) or its derivatives, fluorene or its derivatives, lanthanide complex or its derivatives, squaraine or its derivatives, phthalocyanine or its derivatives, and other organic fluorescent dyes with an absorption wavelength range between 280 nm and 1100 nm that provide a relative high fluorescence wavelength range between 300 nm and 1200 nm. The fluorescent acceptor dyes generally have a relatively high quantum yield (e.g., a quantum yield range between 0.5 to 1.0 for ultraviolet and visual (e.g., blue, yellow-green, red) fluorescent dyes; and a quantum yield range between 0.2 to 0.8 for infrared and near infrared fluorescent dyes) with an emission spectrum having a full width half maximum (FWHM) of less than 65 nm approximately.

Fluorescent acceptor dyes 116 can include reactive groups, including but not limited to, amino, carboxyl or its salt, thiol, alkylhalide, azide, alkyne, aldehyde, maleimide, hydroxyl, boric acid or its ester, acetal, isocyanate, epoxide, acrylate, sulfonate (tosyl, mesyl), nitrophenyl carbonate, Biotins, folic acid, methacrylate, carboxyl, vinyl benzene, mercapto, tetrafluorophenyl esters, succinimidyl ester, pentafluorophenyl ester, hydrazides, vinyl, vinylsulfone, dibenzocyclooctyne group (DBCO), methyltetrazine and other functional organic groups as illustrated in FIG. 6.

In some embodiments, besides reactive groups on fluorescent acceptor dyes 116, previously mentioned, which are already included in one of side groups (R1 to R9) on core of fluorescent acceptors in FIG. 6, the other side groups (R1 to R9) can be independently selected from, but are not limited to, alkyl, substituted alkyl, alkoxy, substituted alkoxy, aryl, substituted aryl, heteroaryl, heterocycle, substituted heteroaryl, polyethylene glycol (PEG) oligomer chain, etc. R1 to R9 side groups may also be one or several fluorescent donor monomers connected with each other via covalent bond. The “M” in FIG. 6 represents different atoms, including but not limited to carbon, silicon, sulfur, nitrogen, germanium, zinc, magnesium, ferric, iron, cobalt, manganese, platinum, silicon, nickel, copper, aluminum, etc.

In some embodiments, the functional groups 118 can include reactive groups which could further react with activated biomolecules, such as proteins, nucleic acids, or carbohydrates.

In some embodiments, functional groups 118 with reactive groups, can be oligomer chain with one functional group, or oligomer chain with two functional groups, or branched oligomers with multi-functional groups. Backbone of oligomer chain include but not limited to alkyl chain, peptide chain, polyethylene oxide chain, etc. The number of repeated units in linker molecules can be from 1 to 10,000. In some embodiments, the number of repeated units in linker molecules ranges from 1 to 5,000. In some embodiments, the number of repeated units in linker molecules ranges from 1 to 4,000. In some embodiments, the number of repeated units in linker molecules ranges from 1 to 2,000. In some embodiments, the number of repeated units in linker molecules ranges from 1 to 1,000. In some embodiments, the number of repeated units in linker molecules ranges from 1 to 500. In some embodiments, the number of repeated units in linker molecules ranges from 1 to 200. In some embodiments, the number of repeated units in linker molecules ranges from 1 to 100. In some embodiments, the number of repeated units in linker molecules ranges from 1 to 50 as illustrated in FIG. 7A-7C.

Referring now to FIGS. 7A-7C, examples of functional groups (functional monomers) 118 are shown. In some embodiments, functional groups 118 with reactive groups include but are not limited to mono-reactive monomers or oligomers, di-reactive monomers or oligomers, tri-reactive monomers or oligomers, in some details, above functional monomers or oligomers have reactive groups at one end that can react with antibody or other bioactive molecules, the reactive group can be thiol, hydroxyl, carboxyl, azide, maleimide, alkyne, biotin, silane, bicyclo[6.1.0]nonyne, dibenzocyclooctyne (DBCO), methyltetrazine, trans-cycloctene (TCO), tetrazine, amino, tetrafluorophenyl esters, succinimidyl ester, pentafluorophenyl ester, bromo, iodol, hydrazide, tosyl, aldehyde, isocyanate, vinyl, vinyl benzene, epoxide, acrylate, etc. functional molecules also has reactive group at the other end that can react with reactive groups on polymer backbone, the reactive group can be amino, thiol, alkyl, halide, Azides, Sulfonic Acid, alkyne, Phenol, aldehyde, maleimide, hydroxyl, Nitrile, Carboxylic acid, or its ester, or its salts, acetal, isocyanate, epoxide, acrylate, alkyl halides, boric acid or its ester, aryl halides, sulfonate (tosyl, mesyl), nitrophenyl carbonate, Biotins, folic acid, methacrylate, mercapto, tetrafluorophenyl esters, succinimidyl ester, pentafluorophenyl ester, hydrazides, vinyl sulfone, dibenzocyclooctyne group (DBCO), methyltetrazine, etc. Functional groups at each side can be different, can also be same.

In some embodiments, mono-functional monomers or oligomers, di-functional monomers or oligomers, tri-functional monomers or oligomers can be used for the reactions respectively at different times. They can also be used for the reactions at the same time.

Generally, the fluorescent non-conjugated nanoparticles can be made by providing a mixture comprising a solvent, said fluorescent polymer dissolved in said solvent, and removing said solvent from said mixture to allow said fluorescent polymer to entangle in the presence of said water, thus forming said water-soluble fluorescent non-conjugated polymer nanoparticles. In some embodiments, an amphiphilic surfactant can be added into the solvent to disperse the nanoparticles. In some embodiments, fluorescent polymeric tandem dye nanoparticles or clusters can be made by injecting fluorescent polymeric tandem dye organic solution into water under sonication, then rotary evaporate to concentrate solution to get nanoparticles aqueous solution. Centrifuge method may also be applied to get concentrated nanoparticles aqueous solution.

In some embodiments, fluorescent polymeric tandem dye nanoparticles or clusters could also be made by injecting fluorescent polymeric tandem dye organic solution into high-speed stirring water solution, then rotary evaporate to concentrate solution to get nanoparticles aqueous solution. Centrifuge method may also be applied to get concentrated nanoparticles aqueous solution.

In some embodiments, solvents for dissolving fluorescent polymeric tandem dyes can be methanol, ethanol, tetrahydrofuran, dimethyl sulfoxide, chloroform, dichloromethane, N,N-dimethylformamide, etc.

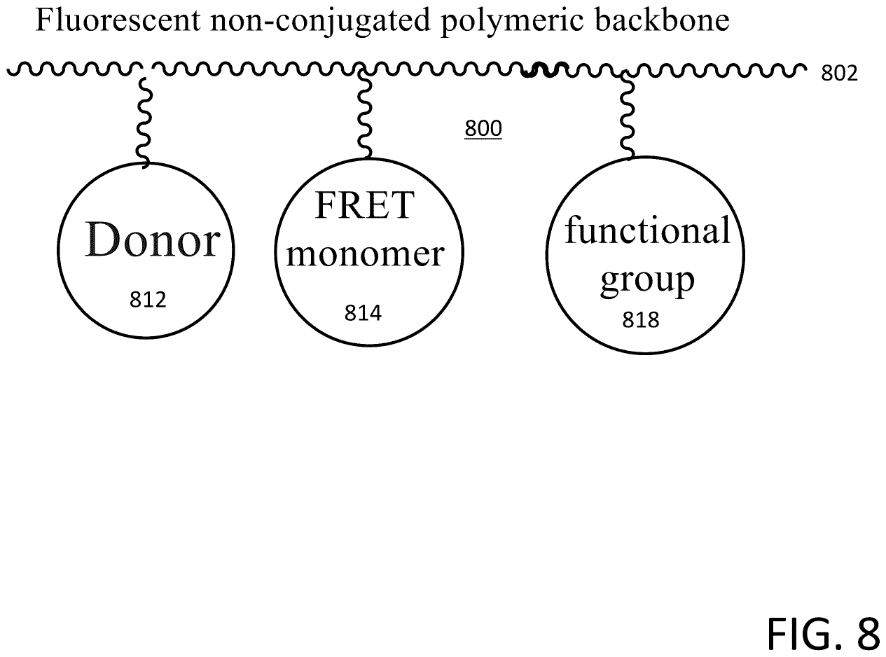

Referring now to FIG. 8, a schematic structure of an embodiment of the fluorescent non-conjugated polymeric tandem dye 800 is illustrated, comprising a fluorescent non-conjugated polymer backbone 802, with sidechains comprising fluorescent donor monomer 812, FRET monomer 814, and functional group 818. In contrast to the fluorescent nonconjugated polymeric tandem dye 100, the fluorescent non-conjugated polymeric tandem dye 800 may comprise fluorescent donor monomer 812, FRET monomer 814, and functional group 818 with the acceptor dye added at a later stage after nano-precipitating and forming nanoparticles is achieved.

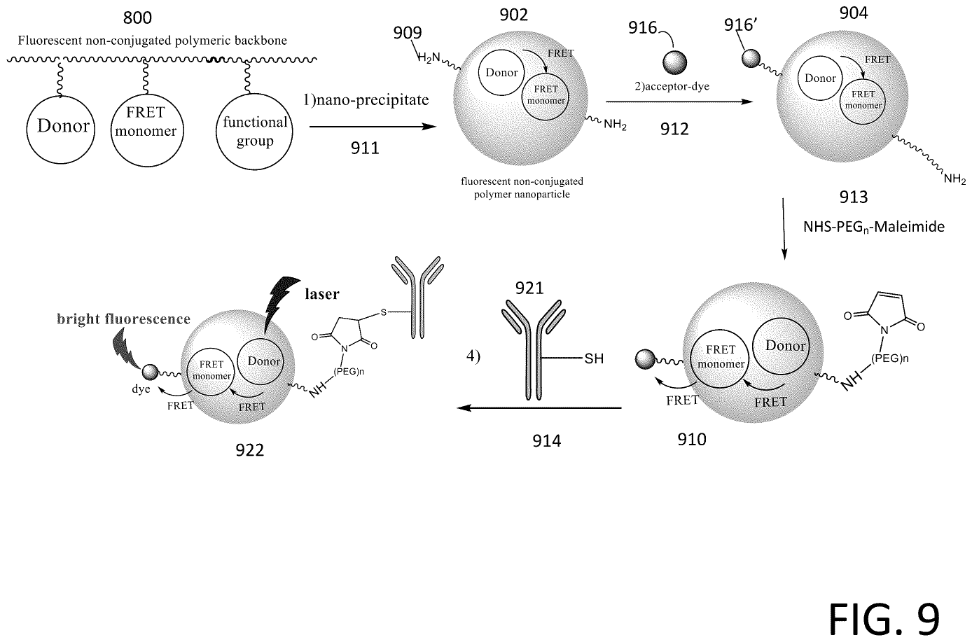

In some embodiments, fluorescent polymeric tandem dye nanoparticles 100 or clusters of the same, can be made by injecting fluorescent non-conjugated polymeric tandem dye 800 organic solution into water under sonication, in which fluorescent non-conjugated polymeric tandem dye 800 only contain fluorescent donor monomers 2012 and FRET monomers 2014, as illustrated in FIG. 8. The organic solution can then be rotary evaporated to concentrate solution to get nanoparticles aqueous solution. Centrifuge method can also be applied to get concentrated nanoparticles aqueous solution. Finally fluorescent acceptor dyes 116 and functional groups 118 can be connected onto nanoparticles by using click chemistry reaction as illustrated in FIG. 9.

FIG. 9 illustrates an exemplary non-conjugated polymer nanoparticle based on the non-conjugated polymer of FIG. 8. FIG. 9 illustrates the formation of the nanoparticle, attaching an acceptor dye to the polymer backbone, adding a water-soluble agent to the nanoparticle, bioconjugating the nanoparticle to an antibody, and exciting the fluorescent donor monomer with a laser causing bright fluorescence at the acceptor dye.

At step 911, the fluorescent non-conjugated polymeric tandem dye 800 shown in FIG. 8 can be introduced to water to nano-precipitate the nanoparticles 902. The nanoparticles 902 can form spherical balls with reactive groups on their surface. The reactive groups on the surface of the nanoparticles 902 can react with fluorescent acceptor dyes 916 and couple them together. In this example, the reactive group is a primary amine 909. The primary amine 909 is a reactive group on the end of the functional group attached to the polymeric backbone. The primary amine (NH₂) 909 can react to reactive groups on the accepter dye 916 to post-graft the accepter dye 916 to the nanoparticle 902.

At step 912, a fluorescent acceptor dye 916 is attached to the surface of the nanoparticles 902 to form fluorescent non-conjugated polymeric tandem dye nanoparticles 904. Previously, a single dye was present in the nanoparticle by the inclusion of the fluorescent donor monomer. The fluorescent acceptor dye 916 bonds to the reactive group on the non-conjugated polymeric backbone 802 to become a pendant fluorescent acceptor dye 916′. The reactive groups react with the primary amine 909 linking the fluorescent acceptor dye 916 to nanoparticle 902 to form the fluorescent non-conjugated polymeric tandem dye nanoparticle 904. The primary amine (NH₂) 909 is positively charged at physiologic pH. The primary amine 909 occurs predominantly on the outside surfaces of native protein tertiary structures where they are readily accessible to reactive groups introduced into the aqueous medium. The primary amine 909 is especially nucleophilic so it is an easy to target for conjugation with several reactive groups.

At step 913, sometimes referred to as a PEGylation process, the fluorescent nonconjugated polymeric tandem dye nanoparticle 904 is further reacted with NHS-PEGn-Maleimide to form a PEGylated fluorescent non-conjugated polymeric tandem dye nanoparticle 910 to prepare for a bioconjugation step to receive an antibody 921. The NHS-PEGn-Maleimide reacts with primary amine 909 to form amide bonds. In some embodiments, the primary amine 909 is a reactive group on the end of the functional group 818 attached to the polymeric backbone. In other embodiments, the primary amine 909 can be a reactive group on the nonconjugated polymeric backbone 802.

NHS-PEGn-Maleimide is often used to prepare antibody conjugates in a two-step reaction. The PEGylation process can also improve the nanoparticle's solubility, stability, and biocompatibility. In this case, the purpose of the reaction in step 913 is to get maleimide groups on the nanoparticles 904 which can further react with thiol (SH) group on the antibody 921 via bioconjugation reaction.

At step 914, the PEGylated fluorescent non-conjugated polymeric tandem dye nanoparticle 910 is further reacted with an antibody 921 to form the bioconjugated fluorescent non-conjugated polymeric tandem dye nanoparticle 922. Typically, the antibody 921 is adapted to bind to specific markers (antigens) on a specific type of biological cells.

Laser energy can then be applied to the bioconjugated fluorescent non-conjugated polymeric tandem dye 922 and excite the fluorescent donor monomer 812, thereby causing a first FRET process to transfer energy from the fluorescent donor monomer 812 to the FRET monomer 814. In turn, the energy transferred to the FRET monomer 814 causes a second FRET process to transfer energy from the FRET monomer 814 to the pendant fluorescent acceptor dye 916′. The pendant fluorescent acceptor dye 916′, can brightly fluoresce due to the efficient energy transfer of energy from the FRET monomer 814 to the pendant fluorescent acceptor dye 916′.

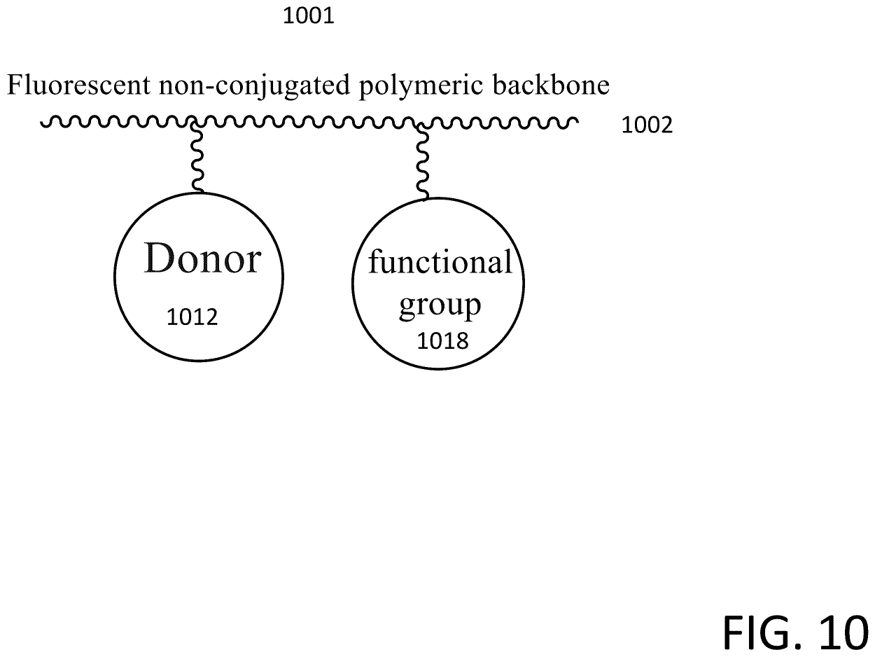

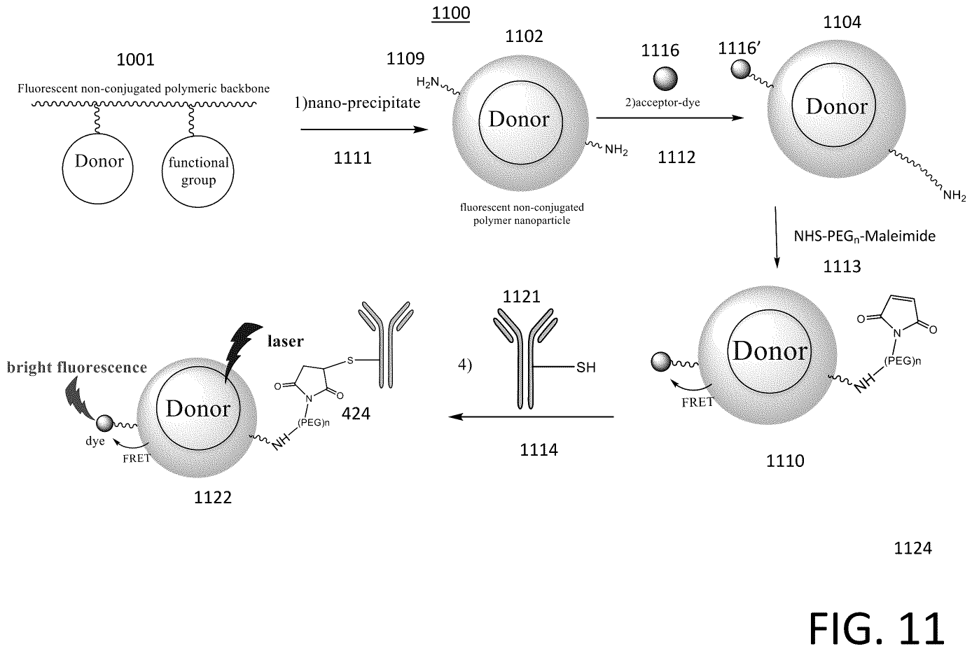

In some embodiments, fluorescent polymeric tandem dye nanoparticles or clusters can be made by injecting fluorescent polymeric dye organic solution into water under sonication, in which fluorescent polymeric dye only contain fluorescent donor monomers, (e.g., see FIG. 10) then evaporate to concentrate solution to get nanoparticles aqueous solution. Centrifuge method may also be applied to get concentrated nanoparticles aqueous solution. Finally fluorescent acceptor dyes and functional groups can be connected onto nanoparticles by using click chemistry reaction (e.g., see FIG. 11).

In summary, a process for preparing fluorescent non-conjugated polymer nanoparticles includes dissolving the fluorescent polymer containing fluorescent donor monomers and FRET monomers into a solvent; injecting the dissolved fluorescent polymer into water under sonication to form a solution; concentrating the solution to get nanoparticles in an aqueous solution; and coupling the fluorescent acceptor dye onto the nanoparticles. Prior to the coupling, an amphiphilic surfactant can be added into the concentrated solution to disperse the nanoparticles. The concentrating of the solution can be performed by rotary evaporation or centrifuging. The fluorescent acceptor dye with the reactive groups can be post-grafted onto the polymer nanoparticles via click chemistry reaction or other type of reaction forming post-grafted fluorescent acceptor dyes. Similarly, molecules of the functional groups are post-grafted onto the polymer nanoparticles via click chemistry reaction or other type of reaction. The solvent can be selected from a group of solvents consisting of methanol, ethanol, tetrahydrofuran, dimethyl sulfoxide, chloroform, dichloromethane, and N, N-dimethylformamide.

Referring now to FIG. 10, a schematic structure of a fluorescent non-conjugated polymer 1001 is shown as yet another disclosed embodiment. The fluorescent non-conjugated polymer 1001 comprises a non-conjugated polymeric backbone 1002 with a fluorescent donor monomer 1012 and a functional group 1018 attached as sidechains to the non-conjugated polymeric backbone 1002. The fluorescent donor monomer 1012 can be similar to the fluorescent donor monomers 112,812. The functional group 1018 can be similar to the functional groups 118,818.

FIG. 11 illustrates exemplary fluorescent non-conjugated polymer nanoparticles based on the fluorescent non-conjugated polymer 1001 shown in FIG. 10. FIG. 11 illustrates the formation of a nanoparticle, attaching an acceptor dye to the polymer backbone, adding a water-soluble agent to the nanoparticle, bioconjugating the nanoparticle to an antibody, and then exciting the fluorescent donor monomer with a laser causing bright fluorescence at the acceptor dye.

At step 1111, the fluorescent non-conjugated polymer 1001 is nano-precipitated to form a fluorescent non-conjugated polymer nanoparticle 1102. Nano-precipitation can be achieved by injecting the fluorescent non-conjugated polymer 1001 into water under sonification. The fluorescent non-conjugated polymer 1001 forms a sphere when nano-precipitated and becomes the fluorescent non-conjugated polymer nanoparticle 1102. Reactive groups are coupled to the surface of the fluorescent non-conjugated polymer nanoparticle 1102.

At step 1112, a reactive group reacts with a fluorescent acceptor dye 1116 is coupled to the fluorescent non-conjugated polymer nanoparticle 1102 forming a fluorescent nonconjugated polymer tandem dye nanoparticle 1104 having a pendant fluorescent acceptor dye 1116′. Previously, a single dye was present in the nanoparticle by the inclusion of the fluorescent donor monomer. The fluorescent acceptor dye 1116 bonds to the reactive group on the non-conjugated polymeric backbone 1002 to become a pendant fluorescent acceptor dye 1116′. The reactive groups react with the amino group 1109 linking the fluorescent acceptor dye 1116 to nanoparticle 1102 to form the fluorescent non-conjugated polymeric tandem dye nanoparticle 1104. The amino group (NH₂) 1109 is positively charged at physiologic pH. The amino group 1109 occurs predominantly on the outside surfaces of native protein tertiary structures where they are readily accessible to reactive groups introduced into the aqueous medium. An amino group 1109 is especially nucleophilic so it is an easy to target for conjugation with several reactive groups.

At step 1113, the fluorescent non-conjugated polymer nanoparticle 1104 is in turn reacted with NHS-PEGn-Maleimide to form a PEG fluorescent non-conjugated polymer tandem dye nanoparticle 1110 having the pendant fluorescent acceptor dye 1116′. The PEG fluorescent non-conjugated polymer nanoparticle 1110 is formed to prepare for a bioconjugation step to receive an antibody 1121. NHS-PEGn-Maleimide is often used to prepare antibody conjugates in a two-step reaction. The NHS-PEGn-Maleimide reacts with the amino group 1109 to form amide bonds.

At step 1114, the PEG fluorescent non-conjugated polymer tandem dye nanoparticle 1110 is bioconjugated to an antibody 1121 forming a bio-conjugated fluorescent non-conjugated polymer tandem dye nanoparticle 1122. Typically, the antibody 1121 is adapted to bind to specific markers (antigens) on a specific type of biological cells.

A laser light from a laser can be used to apply energy to the bio-conjugated fluorescent non-conjugated polymer tandem dye nanoparticle 1122 and excite the fluorescent donor monomer 1012 thereof. The excitement of the fluorescent donor monomer 1012 causes a FRET process to transfer energy from the fluorescent donor monomer 1012 to the pendant fluorescent acceptor dye 1116′ of the bio-conjugated fluorescent non-conjugated polymer tandem dye nanoparticle 1122. The energy transfer excites the pendant fluorescent acceptor dye 1116′ to fluoresce and release a bright fluorescent light (fluorescence) out from the bio-conjugated fluorescent non-conjugated polymer tandem dye nanoparticle 1122.

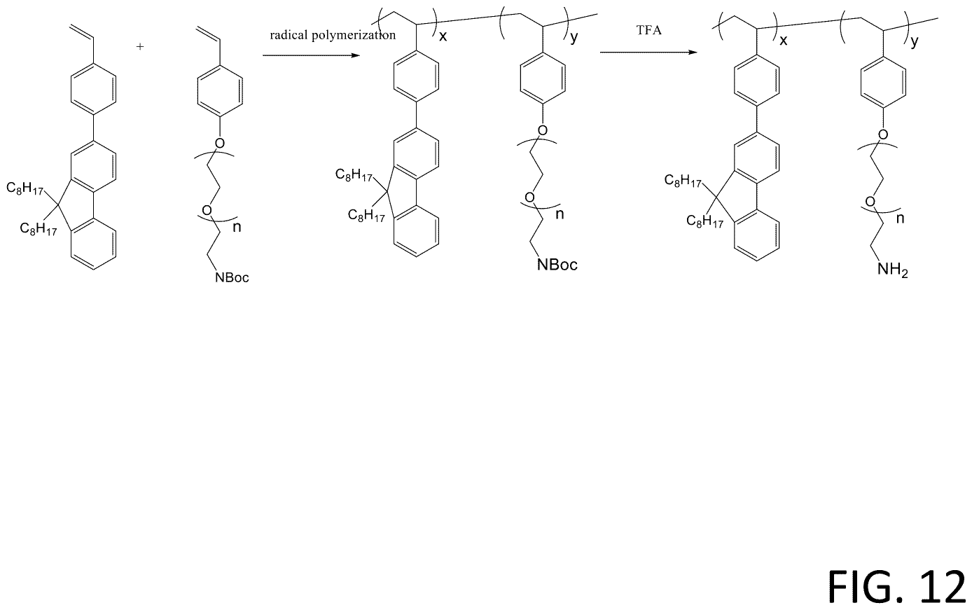

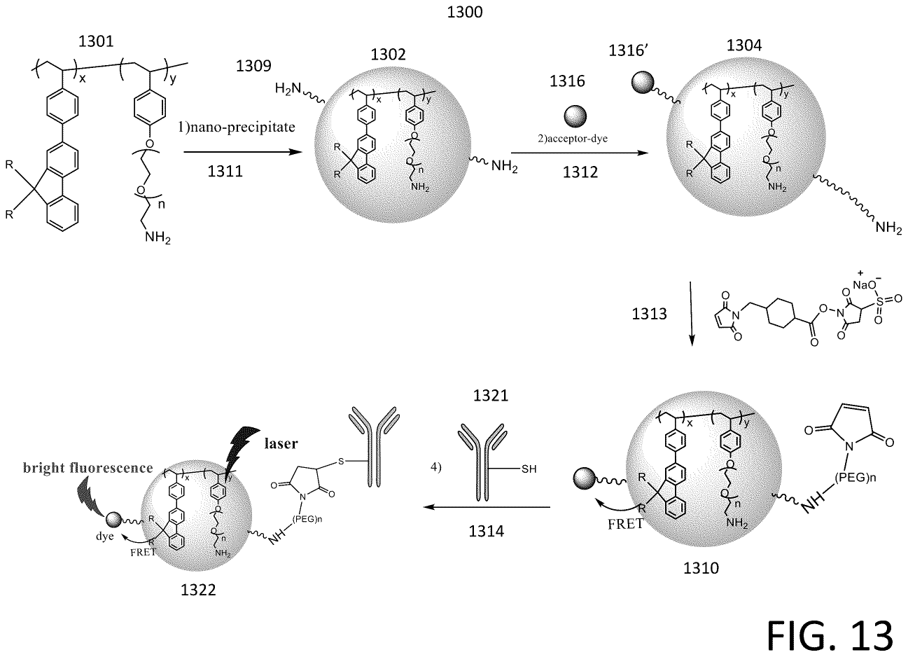

FIG. 12 illustrates formation of a deep ultra-violet excitable fluorescent nonconjugated polymer that can be used to form a nanoparticle. A free radical polymerization process can be used to form a deep ultra-violet (UV) excitable donor polymer that attaches to a non-conjugated backbone to form the deep ultra-violet excitable fluorescent non-conjugated polymer (see also deep ultra-violet (UV) excitable fluorescent non-conjugated polymer 1301 shown in FIG. 13). The fluorescent donor monomers can be converted into fluorescent donor polymers. Twenty-five (25) milliliters (ml) of 4-fluorene styrene and 4,4,5,5-Tetramethyl-1,3,2-dioxaborolane-PEGnNBoc is added into a flask. Two (2) ml of N-Methyl-2-pyrrolidone are added to form a mixture and dissolve the monomers. The containment system is degassed and refilled with Argon. Azobisisobutyronitrile (AIBN) is then added to the mixture. The mixture is then heated at 75 degrees centigrade for 24 hours. The mixture is then washed using dichloromethane and brine to get polymers. The polymers are dissolved in dichloromethane, and trifluoroacetic acid (TFA) is added to deprotect the Boc groups. Finaly, fluorescent donor polymers are obtained after a final washing of the mixture with a solution of brine.

Referring now to FIG. 13, a process for forming bio-conjugated deep ultra-violet (UV) excitable fluorescent non-conjugated polymer nanoparticles 1322 is illustrated based on the formation process of the deep ultra-violet excitable fluorescent non-conjugated polymer shown in FIG. 12. In a series of reactions similar to those illustrated in FIG. 11, the formation of a bio-conjugated deep ultra-violet (UV) excitable fluorescent non-conjugated polymer nanoparticle 1322 starts with the deep ultra-violet (UV) excitable fluorescent non-conjugated polymer 1301. The formation of the deep ultra-violet (UV) excitable fluorescent non-conjugated polymer 1301 includes a fluorescent donor polymer.

At step 1311, the deep ultra-violet (UV) excitable fluorescent non-conjugated polymer 1301 is nano-precipitated to form a fluorescent non-conjugated polymer nanoparticle 1302 with reactive groups coupled to its surface. The reactive groups on the surface of the nanoparticles 1302 can react with fluorescent acceptor dyes 1316 and couple them together.

At step 1312, a reactive group on the nanoparticle 1302 reacts with a fluorescent acceptor dye 1316 to couple the acceptor dye to the nanoparticle, thereby forming a fluorescent non-conjugated polymer tandem dye nanoparticle 1304 with a pendant fluorescent acceptor dye 1316′. Previously, a single dye was present in the nanoparticle 1302 by the inclusion of the fluorescent donor polymer in the deep ultra-violet (UV) excitable fluorescent non-conjugated polymer 1301. The fluorescent acceptor dye 1316 bonds to the reactive group on the nonconjugated polymeric backbone to become a pendant fluorescent acceptor dye 1316′. The reactive groups react with the primary amines 1309 linking the fluorescent acceptor dye 1316 to nanoparticle 1302 to form the fluorescent non-conjugated polymeric tandem dye nanoparticle 1304. An amino group (NH₂) 1309 is positively charged at physiologic pH. The amino group 1309 occurs predominantly on the outside surfaces of native protein tertiary structures where they are readily accessible to reactive groups introduced into the aqueous medium. An amino group 1309 is especially nucleophilic so it is an easy to target for conjugation with several reactive groups.

At step 1313, sometimes referred to as a PEGylation process, the fluorescent nonconjugated polymer tandem dye nanoparticle 1304 is in turn reacted with NHS-PEGn-Maleimide forming a PEG fluorescent non-conjugated polymer tandem dye nanoparticle 1310 to prepare for a bioconjugation step to receive an antibody 1321. The PEG fluorescent non-conjugated polymer tandem dye nanoparticle 1310includes the pendant fluorescent acceptor dye 1316′. The NHS-PEGn-Maleimide reacts with amino group 1309 to form amide bonds. NHS-PEGn-Maleimide is often used to prepare antibody conjugates in a two-step reaction. The PEGylation process can also improve the nanoparticle's solubility, stability, and biocompatibility.

At step 1314, the PEG fluorescent non-conjugated polymer tandem dye nanoparticle 1310 is bioconjugated to an antibody 1321 forming the bio-conjugated deep ultra-violet (UV) excitable fluorescent non-conjugated polymeric tandem dye nanoparticle 1322. Typically, the antibody 1321 is adapted to bind to specific markers (antigens) on a specific type of biological cells.

A laser light from a laser can be used to apply energy to the bio-conjugated deep ultra-violet (UV) excitable fluorescent non-conjugated polymer tandem dye nanoparticle 1322 and excite the fluorescent donor polymer therein. The excitement of the fluorescent donor polymer causes a FRET process to transfer energy from the fluorescent donor polymer to the pendant fluorescent acceptor dye 1316′ of the bio-conjugated deep ultra-violet (UV) excitable fluorescent non-conjugated polymer tandem dye nanoparticle 1322. The energy transfer by the FRET process excites the pendant fluorescent acceptor dye 1316′ to fluoresce and release a bright fluorescent light (fluorescence) out from the bio-conjugated deep ultra-violet (UV) excitable fluorescent non-conjugated polymer tandem dye nanoparticle 1322.

A flow cytometry density plot (dot plot) is a graph that shows the distribution of cells within a population by displaying two parameters as a frequency distribution. Each dot on the density plot represents an individual cell that has passed through the flow cytometer. The color of the dots on the density plot indicates the frequency of events. Using the visible spectrum as an inverted rubric, high density is indicated towards the red, whereas lower densities occupy the green and blue end of the spectrum.

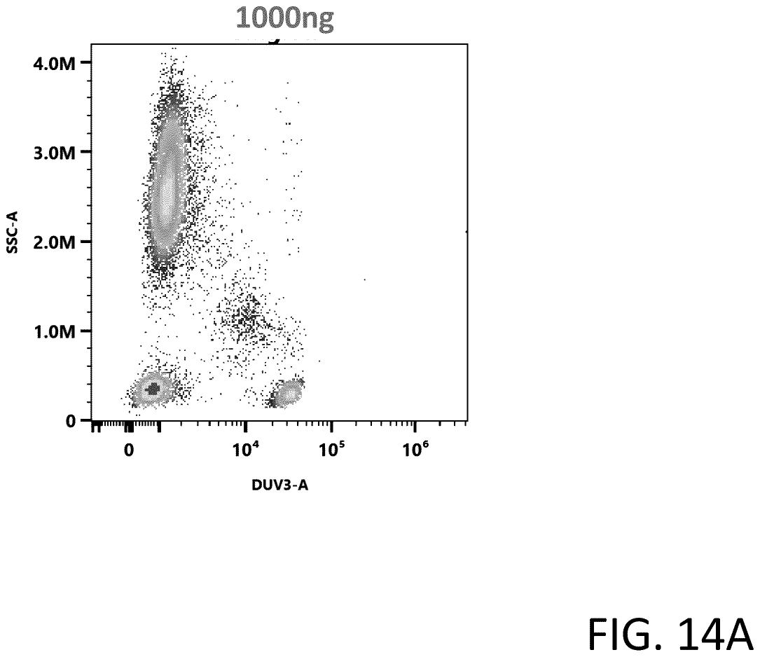

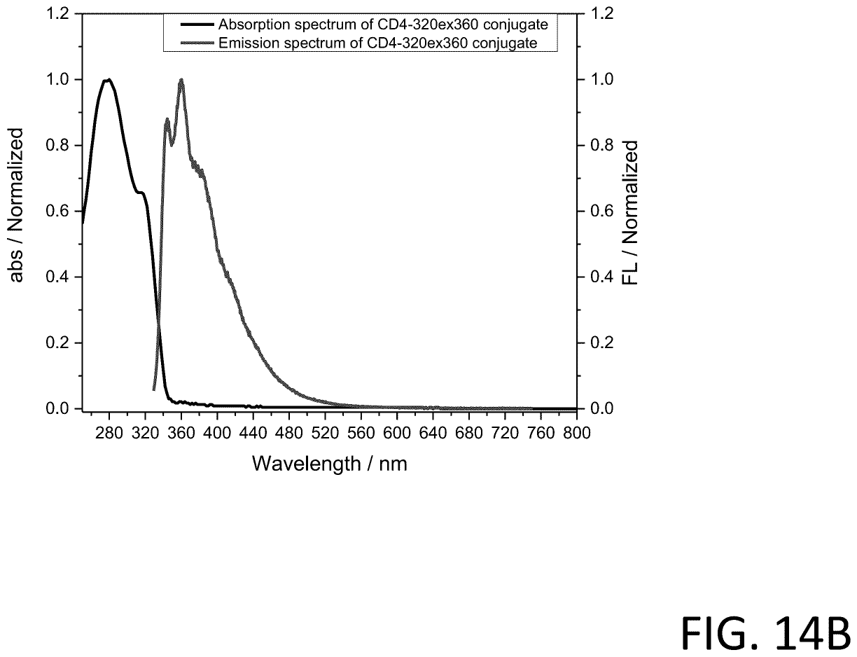

FIGS. 14A, 15A, 16A, 17A, and 18A are density plots obtained after staining sample blood with various embodiments of fluorescent non-conjugated polymer tandem dye nanoparticles and running them through a flow cytometer that uses lasers to excite the fluorescent donor monomer and the pendant fluorescent accepter dye through the FRETT process. Each density plot has a side scatter (SSC) event (population) plotted along the Y axis and light intensity of an ultraviolet detector channel (e.g., DUV3,DUV4,DUV5) along the X axis.

FIGS. 14B, 15B, 16B, 17B, and 18B are charts of emission and absorption spectrum for various embodiments of fluorescent non-conjugated polymer tandem dye obtained by running them through a flow cytometer that uses lasers to excite the fluorescent donor monomer and the pendant fluorescent accepter dye through the FRETT process. Absorption spectrum and emission spectrum are progressively shifted further (i.e., greater Stokes shift), by a FRETT monomer and other means with the FRETT processes, as shown in the charts of FIGS. 14B, 15B, 16B, 17B, and 18B. A single laser generating a laser light with the same excitation wavelength (e.g., 320 nm center wavelength) is used to excite the tandem dye which is absorbed by the fluorescent donor and thereby excites the acceptor dye at the various center wavelengths.

### CD 4-320ex360 at 1000 Ng

FIG. 14A illustrates an exemplary SSC vs. deep ultra-violet channel DUV3 density plot. (whole blood cells were stained with CD4-320ex360 at 1000 nanograms (ng) FIG. 14B illustrates absorption and fluorescence spectra of CD4-320ex360 conjugate in PBS buffer.

### Bioconjugation between NCP-320ex360 and Activated Antibody

0.5 milligrams (mg) or 1000 ng of CD4 antibody was activated with DL-Dithiothreitol (DTT) at room temperature for 1 hr, then use a NAP-5 column to separate the reduced IgG from free DTT. At the same time, NCP-320ex360 react Sulfo-SMCC (sulfosuccinimidyl 4-(N-maleimidomethyl) cyclohexane-1-carboxylate) at room temperature for 1 hr. then use a NAP-10 column to separate the activated NCP-320ex360(320ex360-maleimide) from the free SMCC. In a container, mix the NCP320ex360-maleimide solution with the reduced CD4 solution dropwise, allow to react at room temperature for 2 hrs. 2-mercaptoethanol was added to stop the reaction. Conjugate was purified using Fast Protein Liquid Chromatography (FPLC).

Full spectrum flow cytometry test of CD4-320ex360 bioconjugates were used to label whole blood cells. FIG. 14A show the full spectrum flow cytometry results, which proved that CD4-320ex360 probes were effectively labeled on the cell surface and gave a brighter signal. FIG. 14B show absorption and fluorescence spectra of CD4-320ex360 bioconjugates.

### CD4-320ex372 at 1000 Ng

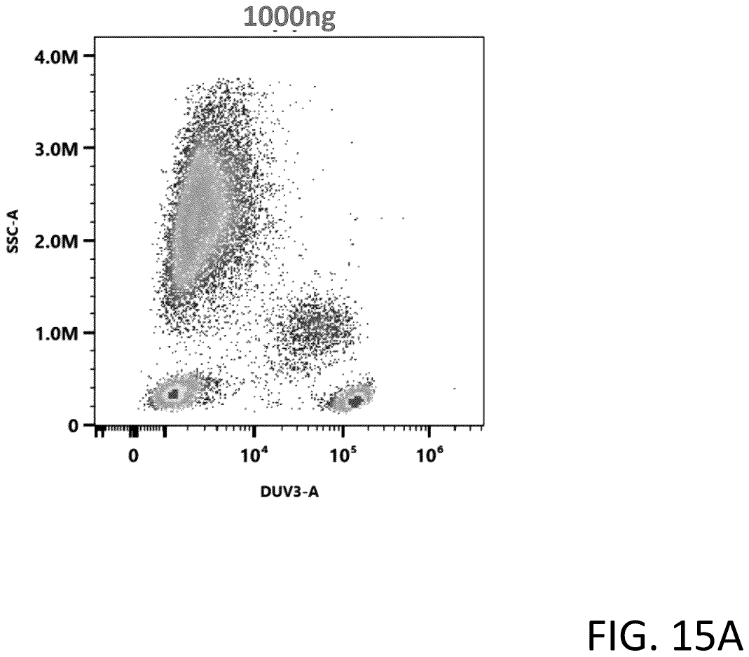

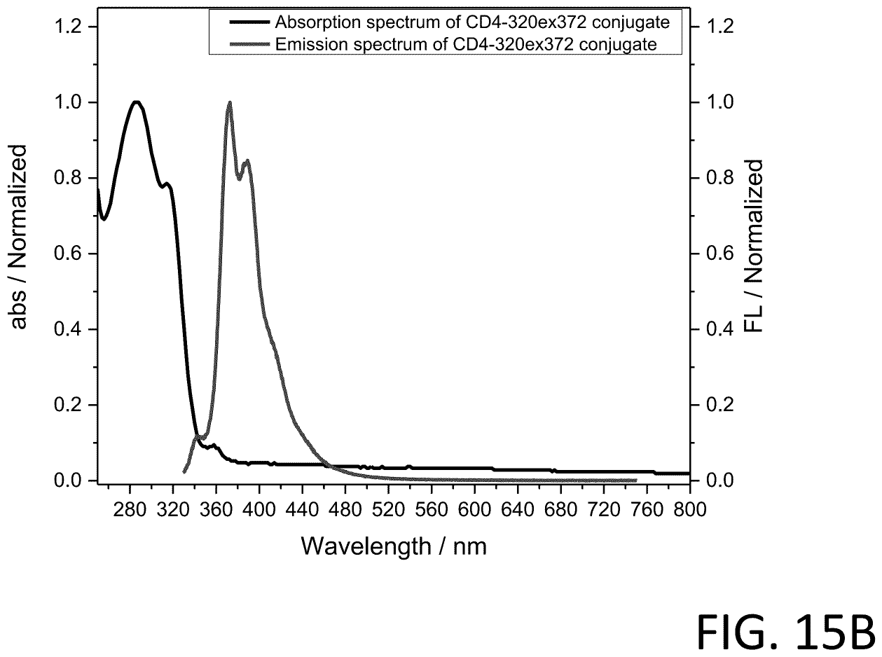

FIG. 15A illustrates an exemplary SSC vs. Deep ultra-violet channel DUV3 density plot. (whole blood cells were stained with CD4-320ex372 at 1000 ng) FIG. 15B illustrates an exemplary absorption and fluorescence spectra of CD4-320ex372 conjugate in PBS buffer.

### Bioconjugation between NCP-320ex372 and Activated Antibody

0.5 mg (1000 ng) of CD4 antibody was activated with DL-Dithiothreitol (DTT) at room temperature for 1 hr, then use a NAP-5 column to separate the reduced IgG from free DTT. At the same time, NCP-320ex372 react Sulfo-SMCC (sulfosuccinimidyl 4-(N-maleimidomethyl) cyclohexane-1-carboxylate) at room temperature for 1 hr. then use a NAP-10 column to separate the activated NCP-320ex372 (320ex372-maleimide) from the free SMCC. In a container, mix the NCP320ex372-maleimide solution with the reduced CD4 solution dropwise, allow to react at room temperature for 2 hrs. 2-mercaptoethanol was added to stop the reaction. Conjugate was purified using Fast Protein Liquid Chromatography (FPLC).

Full spectrum flow cytometry test CD4-320ex372 bioconjugates were used to label whole blood cells. FIG. 15A show the full spectrum flow cytometry results, which proved that CD4-320ex372 probes were effectively labeled on the cell surface and gave a brighter signal. FIG. 15B show absorption and fluorescence spectra of CD4-320ex372 bioconjugates.

### CD4-320ex385 at 1000 Ng

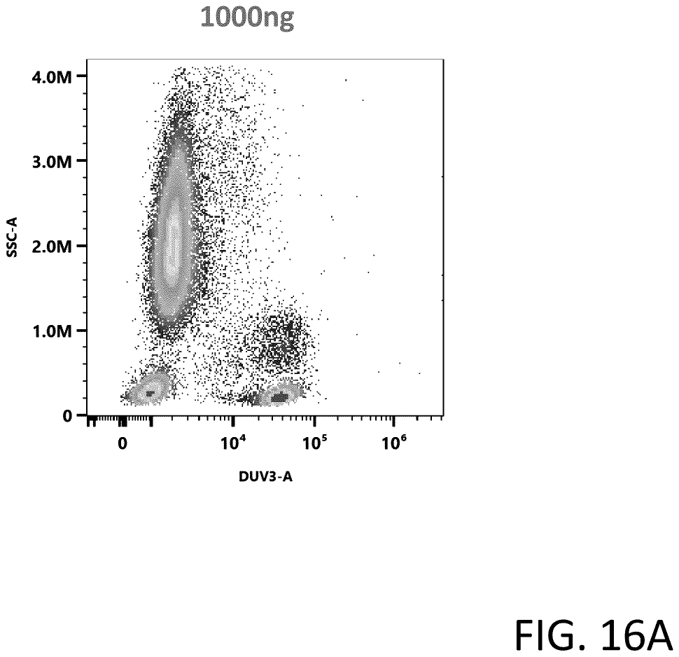

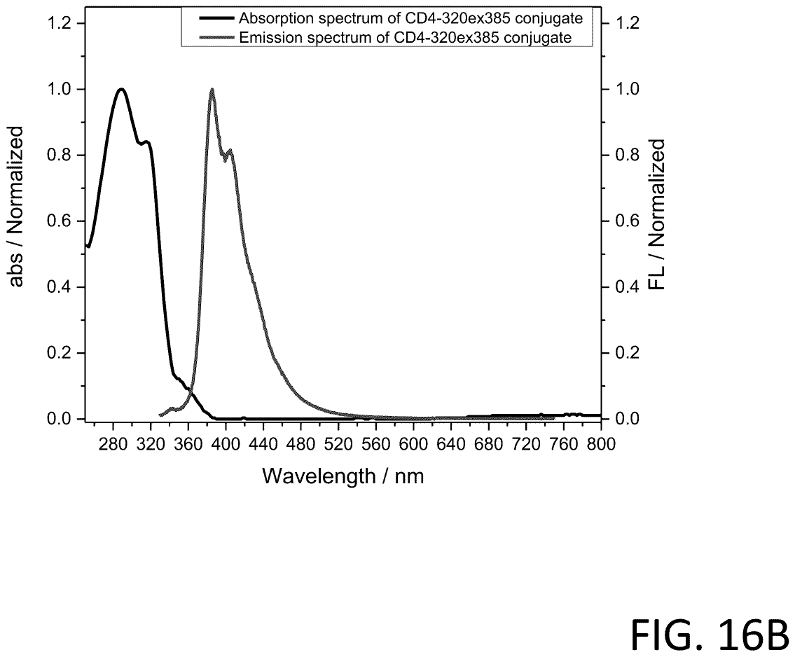

FIG. 16A illustrates an exemplary SSC vs. Deep ultra-violet channel DUV3 density plot. (whole blood cells were stained with CD4-320ex385 at 1000 ng) FIG. 16B illustrates an exemplary absorption and fluorescence spectra of CD4-320ex385 conjugate in PBS buffer. One thousand nanograms of CD4-320ex385 conjugate are used to label the cell to run the flow cytometry test to obtain the results shown in FIG. 16A.

### Bioconjugation between NCP-320ex385 and Activated Antibody

One half milligram (0.5 mg) (500 ng) of CD4 antibody was activated with DL-Dithiothreitol (DTT) at room temperature for 1 hr, then use a NAP-5 column to separate the reduced IgG from free DTT. At the same time, NCP-320ex385 react Sulfo-SMCC (sulfosuccinimidyl 4-(N-maleimidomethyl) cyclohexane-1-carboxylate) at room temperature for one hour. Then NAP-10 column is used to separate the activated NCP-320ex385 (320ex385-maleimide) from the free SMCC. In a container, mix the NCP320ex385-maleimide solution with the reduced CD4 solution dropwise, and allow the mixture to react at room temperature for two hours to form a conjugate. The chemical 2-mercaptoethanol is then added to stop the reaction. The conjugate is then purified using a Fast Protein Liquid Chromatography (FPLC) step.

Full spectrum flow cytometry test of CD4-320ex385 bioconjugates were used to label whole blood cells. FIG. 16A show the full spectrum flow cytometry results, which proved that CD4-320ex385 probes were effectively labeled on the cell surface and gave brighter signal. FIG. 16B show absorption and fluorescence spectra of CD4-320ex385 bioconjugates.

### CD4-320ex435 at 500 Ng

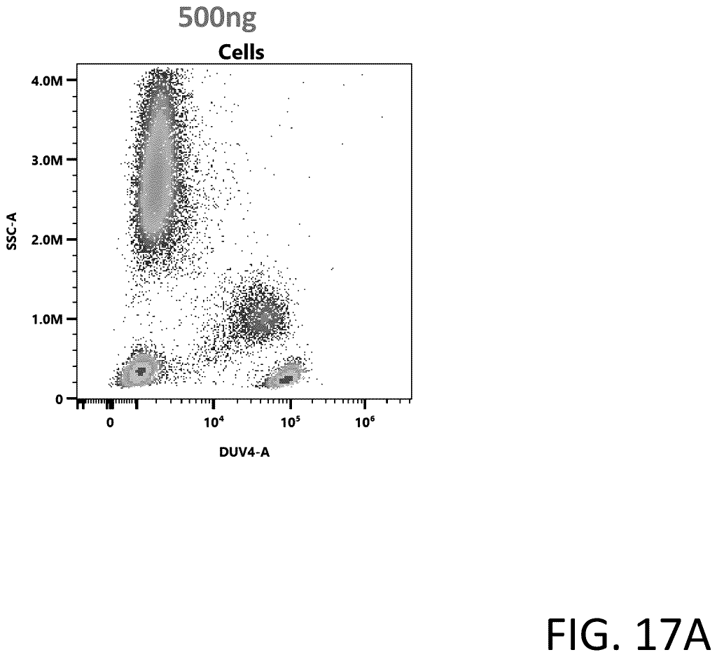

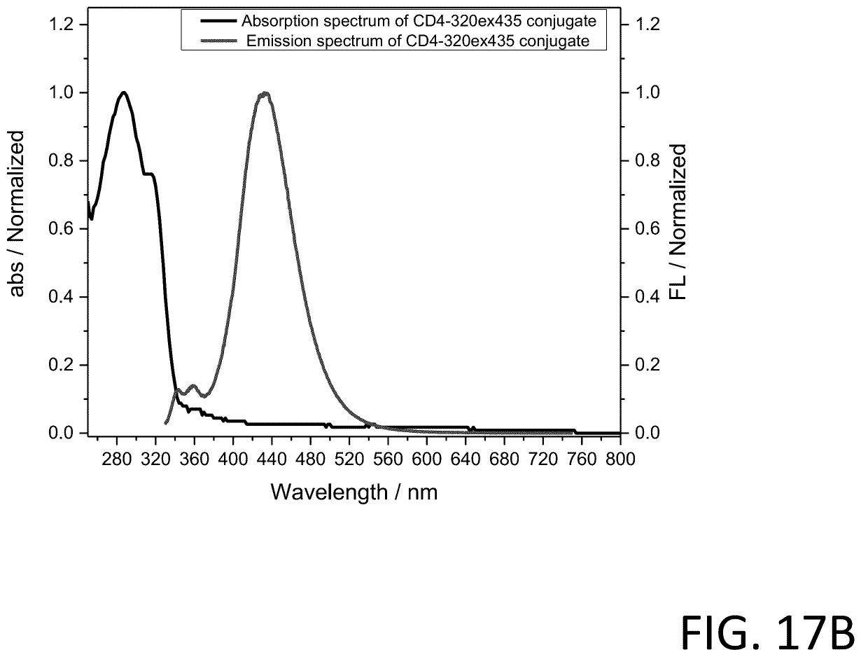

FIG. 17A illustrates an exemplary SSC vs. Deep ultra-violet channel DUV4 density plot. (whole blood cells were stained with CD4-320ex435 at 500 ng)

FIG. 17B illustrates an exemplary absorption and fluorescence spectra of CD4-320ex435 conjugate in PBS buffer.

### Bioconjugation between NCP-320ex435 and Activated Antibody

0.5 mg (500 ng) of CD4 antibody was activated with DL-Dithiothreitol (DTT) at room temperature for 1 hr, then use a NAP-5 column to separate the reduced IgG from free DTT. At the same time, NCP-320ex435 react Sulfo-SMCC (sulfosuccinimidyl 4-(N-maleimidomethyl) cyclohexane-1-carboxylate) at room temperature for 1 hr. then use a NAP-10 column to separate the activated NCP-320ex435 (320ex435-maleimide) from the free SMCC. In a container, mix the NCP320ex435-maleimide solution with the reduced CD4 solution dropwise, allow to react at room temperature for 2 hrs. 2-mercaptoethanol was added to stop the reaction. Conjugate was purified using Fast Protein Liquid Chromatography (FPLC).

Full spectrum flow cytometry test of CD4-320ex435 bioconjugates were used to label whole blood cells. FIG. 17A show the full spectrum flow cytometry results, which proved that CD4-320ex435 probes were effectively labeled on the cell surface and gave brighter signal. FIG. 17B show absorption and fluorescence spectra of CD4-320ex435 bioconjugates.

### CD4-320ex457 at 500 Ng

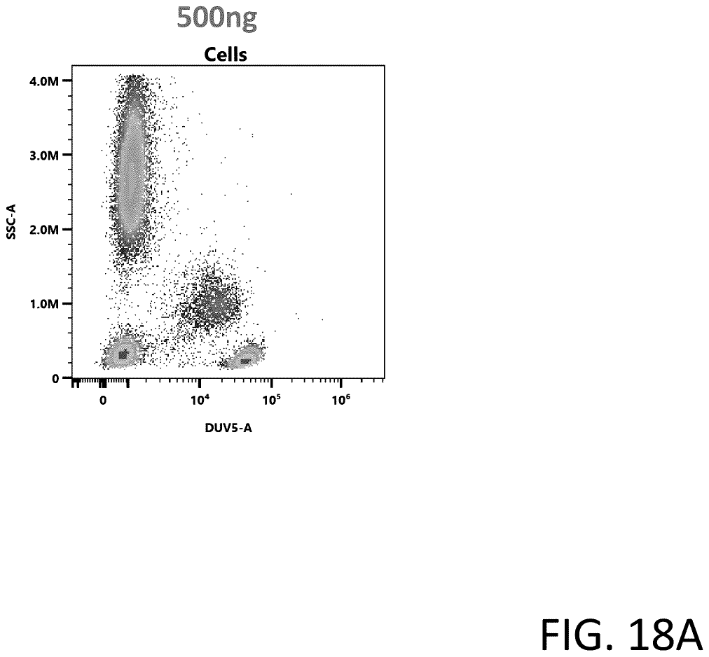

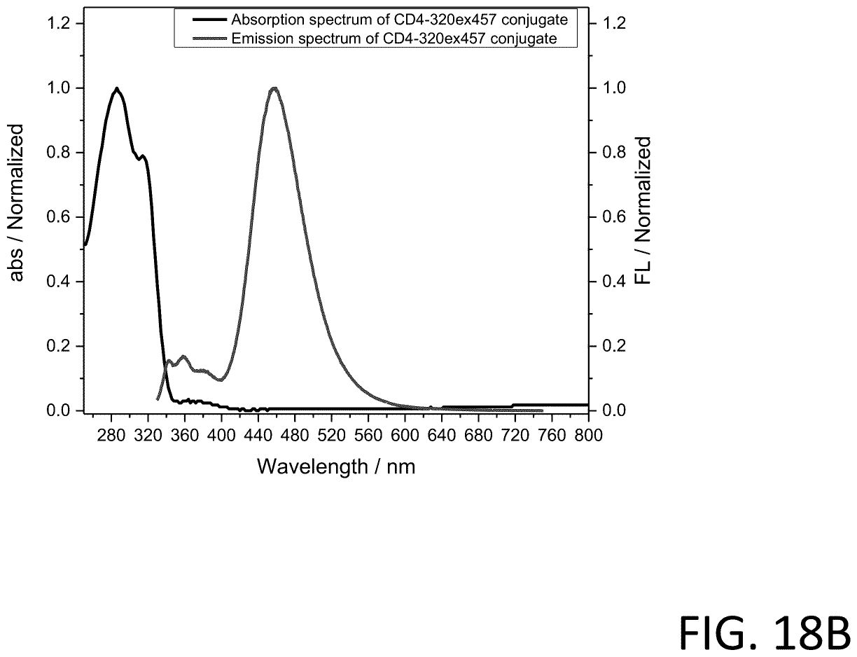

FIG. 18A illustrates an exemplary SSC vs. Deep ultra-violet channel DUV5 density plot. (whole blood cells were stained with CD4-320ex457 at 500 ng)

FIG. 18B illustrates an exemplary absorption and fluorescence spectra of CD4-320ex457 conjugate in PBS buffer.

### Bioconjugation between NCP-320ex457 and activated antibody

0.5 mg (500 ng) of CD4 antibody was activated with DL-Dithiothreitol (DTT) at room temperature for 1 hr, then use a NAP-5 column to separate the reduced IgG from free DTT. At the same time, NCP-320ex457 react Sulfo-SMCC (sulfosuccinimidyl 4-(N-maleimidomethyl) cyclohexane-1-carboxylate) at room temperature for 1 hr. then use a NAP-10 column to separate the activated NCP-320ex457 (320ex457-maleimide) from the free SMCC. In a container, mix the NCP320ex457-maleimide solution with the reduced CD4 solution dropwise, allow to react at room temperature for 2 hrs. 2-mercaptoethanol was added to stop the reaction. Conjugate was purified using Fast Protein Liquid Chromatography (FPLC).

Full spectrum flow cytometry test of CD4-320ex457 bioconjugates were used to label whole blood cells. FIG. 18A show the full spectrum flow cytometry results, which proved that CD4-320ex457 probes were effectively labeled on the cell surface and gave a brighter signal. FIG. 18B show absorption and fluorescence spectra of CD4-320ex457 bioconjugates.

The embodiments have a number of advantages. The tandem dyes use an efficient intra-nanoparticle FRET process to transfer energy from the donor dye to the acceptor dye, providing a greater intensity or brighter fluorescence. The tandem dyes and FRET processes can provide greater light intensity for improved detection in a flow cytometer using a single laser. The tandem dyes and FRET processes can also provide a greater stokes shift to separate the excitation wavelength from the absorption wavelength to provide different emission spectra for improved detection in a flow cytometer. The nanoparticle structure of the tandem dyes can reduce interchain entanglement to improve FRET energy transfer efficiency, reducing erroneous energy transfer. to provide narrower bandwidth of emission spectra. The nanoparticle structure of the tandem dyes can increase intrachain entanglement thereby increasing absorption efficiency and thereby increasing emission intensity.

### Packaging into Container

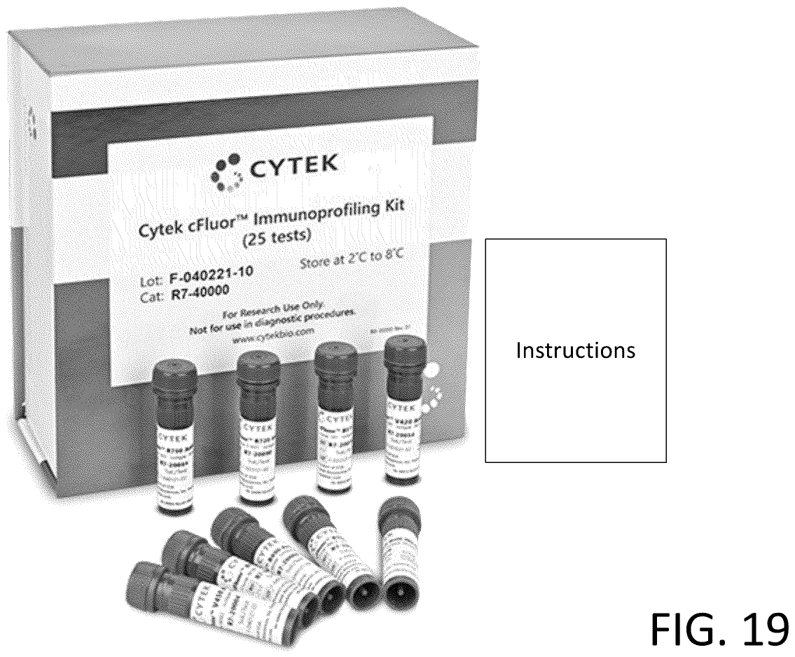

Referring now to FIG. 19, solutions with the disclosed embodiments of the fluorescent non-conjugated polymer nanoparticles bio-conjugated with antibodies to form bio-conjugated reagents that can be shipped in one or more sealable vials in a box with instructions of use. Other reagents can be in other sealable vials in the box to form an immunoprofiling kit. The bio-conjugated reagents can be added into one or more sample test tubes. A biological sample, such as whole blood or peripheral blood mononuclear cell (PBMC), can be added into the one or more sample test tube that can then be run through a spectral flow cytometer to obtain results of cell counts in gated populations and further information (e.g., size, shape, etc.) about the cells in the sample.

### Flow Cytometer and Sample Run

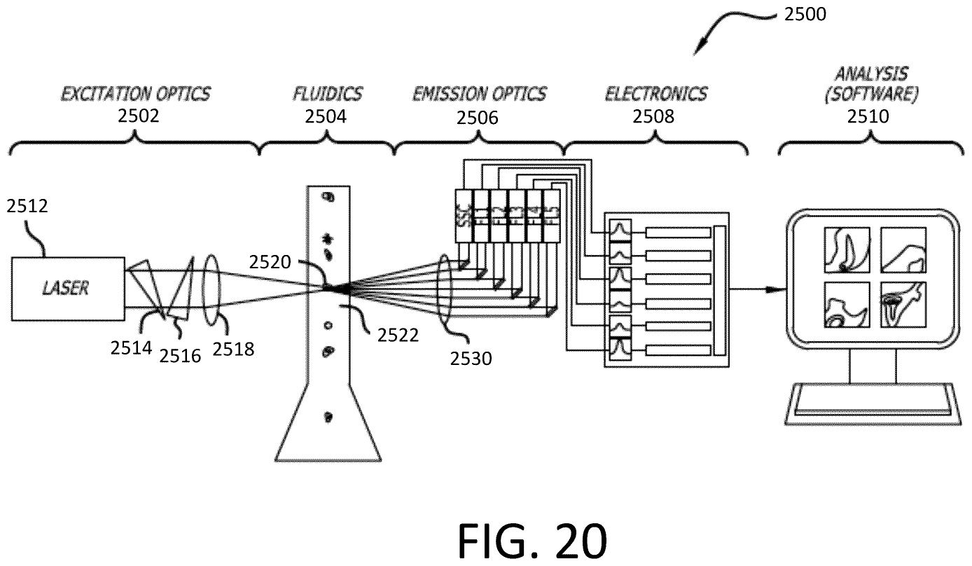

Referring now to FIG. 20, a basic conceptual diagram of a flow cytometer system 2500 is shown. Various embodiments of the flow cytometer 2500 may be commercially available. Five major subsystems of the flow cytometer system 2500 include an excitation optics system 2502, a fluidics system 2504, an emission optics system 2506, an acquisition system 2508, and an analysis system 2510. Generally, a “system” includes hardware devices, software devices, or a combination thereof.

The excitation optics system 2502 includes, for example, a laser device 2512, an optical element 2514, an optical element 2516, and an optical element, 2518. Example optical elements include an optical prism and an optical lens. The excitation optics system 2502 illuminates an optical interrogation region 2520. The fluidics system 2504 carries fluid samples 2522 through the optical interrogation region 2520. The emission optics system 2506 includes, for example, an optical element 2530 and optical detectors SSC, FL1, FL2, FL3, FL4, and FL5. The emission optics system 2506 gathers photons emitted or scattered from passing particles. The emission optics system 2506 focuses these photons onto the optical detectors SSC, FL1, FL2, FL3, FL4, and FL5. Optical detector SSC is a side scatter channel. Optical detectors FL1, FL2, FL3, FL4, and FL5 are fluorescent detectors may include band-pass, or long-pass, filters to detect a particular fluorescence wavelength. Each optical detector converts photons into electrical pulses and sends the electrical pulses to the acquisition system 2508. The acquisition system 2508 processes and prepares these signals for analysis in the analysis system 2510.

The analysis system 2510 can store digital representations of the signals for analysis after completion of acquisition. The analysis system 2510 is a computer with a processor, memory, and one or more storage devices that can store and execute analysis software to obtain laboratory results of biological samples (or other types of samples, e.g., chemical) that are analyzed. The analysis system 2510 can be further used to calibrate the flow cytometer with compensation controls when initialized, before running a reference sample through the flow cytometer. Reference samples can be formed in different ways to determine spillover vectors for a fluorescent dye or fluorochrome. A fluorochrome can be conjugated with an antibody and then attached to a biological cell or attached to a bead or particle.

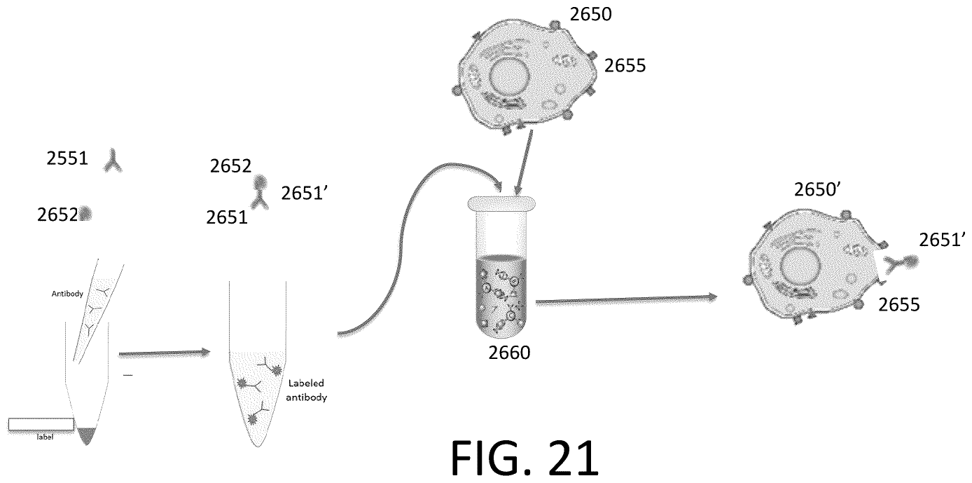

Referring now to FIG. 21, a cell 2650, an antibody 2651, and a fluorochrome (dye) 2652 are coupled together to form a reference sample with direct marking or staining of a cell. The cell 2650 has one or more cell marker 2655 sites to which an antibody can attach. The fluorochrome (dye) 2652 is conjugated with the antibody 2651 in advance to form a conjugated antibody 2651′. For a reference sample, a single fluorochrome (dye) 2652 is conjugated with a single antibody to generate a spillover vector. Subsequently, when analyzing a biological fluid with different unknown counts of cells in the biological fluid, multiple conjugated antibodies with different antibodies and different fluorochrome, can be used and add into the same biological sample.

The conjugated antibodies 2651′ and the cells 2650 are mixed together in a test tube 2660 so the conjugated antibodies 2651′ can attached to the desired cell marker sites 2655 for the given type of cells 2650 to form marked or stained cells 2650′ in the sample biological fluid. When run through the flow cytometer, the fluorochromes can be excited by laser light to fluoresce so that the fluorescence can be detected by detectors as events generating an event vector. The event vector can be used to generate a spill over matrix for the fluorochrome. When running a sample biological fluid with unknown counts, the cells counted by a flow cytometer by analyzing the events.

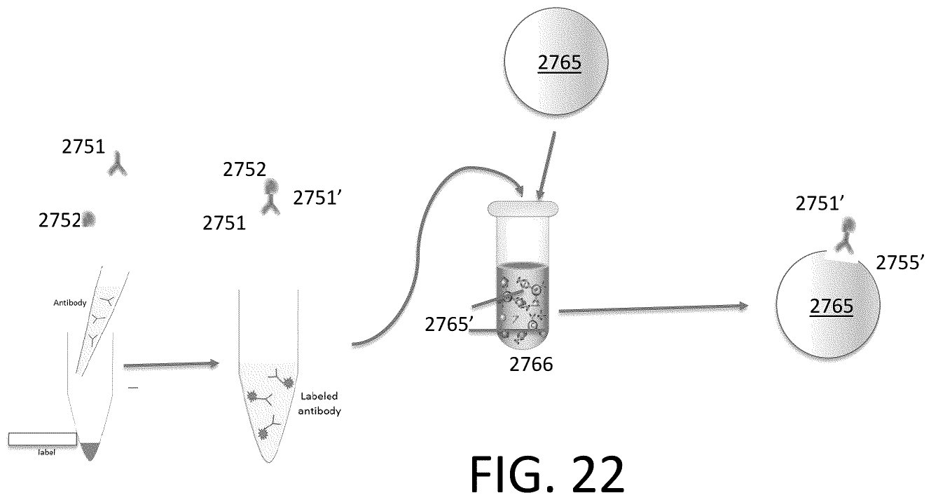

Referring now to FIG. 22, a conceptual diagram of forming a reference sample with a bead 2765 is shown. A bead 2765, an antibody 2751, and a fluorochrome (dye) 2752 are coupled together to form a reference sample with a bead. The bead 2765 may have one or more cell marker 2755′ sites to which an antibody can attach. As with the cell, the fluorochrome (dye) 2752 is conjugated with the antibody 2751 in advance to form a conjugated antibody 2751′. For a reference sample, a single fluorochrome (dye) 2752 is conjugated with a single antibody to generate a spillover vector.

The conjugated antibodies 2751′ and the beads 2765 are mixed together in a test tube 2766 so the conjugated antibodies 2751′ can attached to the desired marker sites 2755′ for the beads 2765 to form marked beads 2765′ in a reference sample. When run through the flow cytometer, the fluorochromes can be excited by laser light to fluoresce so that the fluorescence can be detected by detectors as events generating an event vector. The event vector can be used to generate a spill over matrix for the fluorochrome. In this manner, either cells or beads can be used to test and fluorochrome for suitability to be used with a flow cytometer.

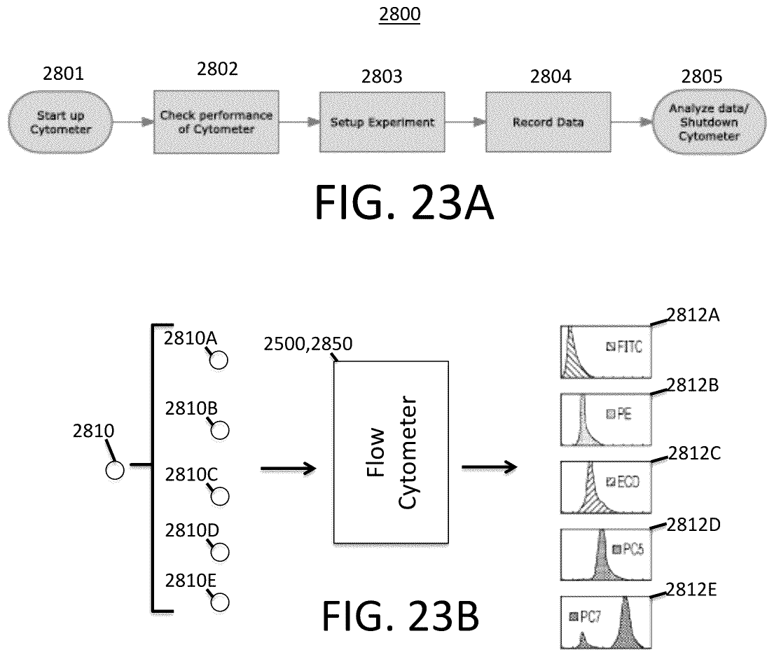

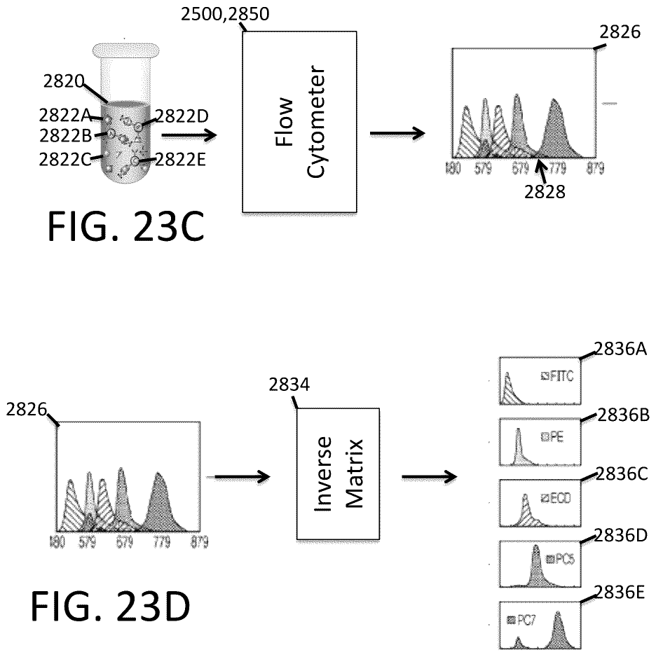

Referring now to FIG. 23A, a flowchart of a method 2800 for a flow cytometer is shown. The flow cytometry system 2500 of FIG. 20, or other flow cytometer systems, can carry out the method 2800. Flow cytometry allows for data collection and analysis of data on single cells or particles of a plurality that are in a sample fluid.

In step 2801, the system starts up the flow cytometer. In step 2802, the system checks the performance of the flow cytometer and performs calibration if and as needed with calibration beads. If the flow cytometer was recently calibrated (e.g., same day or same hour), this step can be skipped.

In step 2803, multiple experiments are setup to run to generate spillover vectors for each dye. A reference sample is prepared (fluorochrome conjugated to an antibody that is attached to a cell or a bead) to initially run to generate event vectors that can be converted into a spillover vector.

In step 2804, the reference sample fluid with one fluorochrome is run through the flow cytometer for analysis with the data captured from N detectors being recorded. Multiple runs through the flow cytometer with the same reference sample fluid may be performed to be sure measurements are well understood. The data from N detectors is recorded for each run of the reference sample through the flow cytometer.

In step 2805, after the sample fluid or calibration beads are run through the flow cytometer, the recorded data can be analyzed to determine results from the analysis by the flow cytometer.

Each spillover vector for one fluorochrome can be subsequently compared with another spillover vector for another fluorochrome to determine how different combinations of pairs of fluorochromes (dyes) and markers interact and spectrally interfere. The spillover vectors for each dye can be subsequently combined together into a spillover matrix for a total number and types of dye being used together to identify cells/particles in a single sample. Combinations of pairs of spillover vectors (columns) in the spillover matrix can be compared together to determine a similarity index between the two fluorochromes. For each reference sample, the light intensity density for each channel can saved as a reference vector and the data can be binned and plotted to form a full spectrum signature for the given fluorochrome.

The flow cytometer can also be shut down if no further samples or calibration beads are to be run. Alternatively, another sample or more calibration beads can be run through the flow cytometer to obtain and record (save) data and subsequently analyze the recorded data.

In step 2805, the system performs single stained compensation controls to generate an initial spillover matrix or reference matrix. When performing multicolor flow cytometry, the system uses single stained samples (reference samples) 2810A-2810E (collectively referred to by reference number 2810) run through a flow cytometer 2500,2850 to determine the levels of compensation, such as shown in FIG. 23B. Single staining of the particles 2810A-2810E can reveal the respective spectral profile or signature 2812A-2812E of respective fluorochromes to the fluorescent photodetectors of the instrument. The information obtained from the single stained particles 2810 can be subsequently used to determine a simplicity index and a complexity index of a set of fluorochromes attached to the particles 2810. The information obtained from the single stained particles 2810 can also be subsequently used to determine a reference full spectrum signature for a fluorochrome useful for unmixing data from a mixed sample labeled with multiple fluorochromes.

The staining of the compensation control usually should be as bright or brighter than the sample. Antibody capture beads can be substituted for cells and one fluorophore conjugated antibody for another, if the fluorescence measured is brighter for the control. The exceptions to this are tandem dyes, which cannot be substituted. Tandem dyes from different vendors or different batches must be treated like separate dyes, and a separate single-stained control should be used for each because the amount of spillover may be different for each of these dyes. Also, the compensation algorithm should be performed with a positive population and a negative population. Whether each individual compensation control contains beads, the cells used in the experiment, or even different cells, the control itself must contain particles with the same level of auto-fluorescence. The entire set of compensation controls may include individual samples of either beads or cells, but the individual samples must have the same carrier particles for the fluorophores. Also, the compensation control uses the same fluorophore as the sample. For example, both green fluorescent protein (GFP) and Fluorescein isothiocyanate (FITC) emit mostly green photons, but have vastly different emission spectra. Accordingly, the system cannot use one of them for the sample and the other for the compensation control. Also, the system must collect enough events to make a statistically significant determination of spillover (e.g., about 5,000 events for both the positive and negative population).

During calibration in a conventional flow cytometer, the system obtains an initial spillover matrix from single stained reference controls. In a conventional flow cytometer, the fluorescence signals (e.g., colors) are separated out into discrete fluorescent bands using a series of edge filters and dichroic mirrors. The system detects (e.g., measures) each individual channel with a photo multiplying tube (PMT). During detection of the fluorescent signals, “spillover” can occur between fluorescent bands, which ideally are completely discrete, such as shown in the combined profile 2826. The system defines the spillover (e.g., spillover 2828 in the combined profile 2826 in FIG. 23C) between the fluorescent bands with a spillover matrix [S].

Alternatively, during calibration in a spectral flow cytometer, the system obtains an initial reference matrix from single stained reference controls 2810. Spectral flow cytometry is a technique based on conventional flow cytometry where a spectrograph and multichannel detector (e.g., charge-coupled device (CCD)) is substituted for the traditional mirrors, optical filters and photomultiplier tubes (PMT) in conventional systems. In the spectral flow cytometer, the side scattered light and fluorescence light is collected and coupled into a spectrograph, either directly or through an optical fiber, where the whole light signal is dispersed and displayed as a high-resolution spectrum on the CCD or coupled into one or more multichannel detectors for detection.

In process step 2804 of FIG. 23A, the sample 2820 shown in FIG. 23C is run through the flow cytometer 2500,2850. The sample 2820 includes a plurality of marked cells or particles 2822A-2822E that flow through each laser beam of each laser and generates fluorescent light and/or scattered light referred to as an event. The fluorescent light and/or scattered light is captured and detected in order to identify the particle and generate counts for the various types of particles in the sample 2820. For each particle in the sample fluid 220 passing by the laser beam(s) and fluorescing light and/or scattering light, the system generates, obtains, and/or records data (e.g., event data) representing the overall spectral profile 2826. For example, fluoresced cells in the sample fluid flowing through the flow cytometer are detected. An event occurs per particle/cell. Each full spectrum detection of a fluoresced cell by the detector modules excited by the lasers is an event. The event data for a particle/cell may be defined according to a measured sample event vector.

In step 2805, the system generates a compensated sample event vector (for conventional flow cytometer) or an unmixed sample event vector (for spectral flow cytometer) to count the number of various types of cells or particles in a sample 2822 to obtain a measure of concentration. Generally as shown in FIG. 23D, an inverse matrix 2834 (determined from the initial spillover matrix and/or the initial reference matrix with fine adjustments) is used on the event data representing the spectral profile 2826 to generate the compensated sample event vector or the unmixed sample event vector representing separate spectral profiles or signatures 2836A-2836E of the various auto-luminescence (generated by the cells or particles themselves) or luminescence given off by the fluorochromes tagged to the various cells 2822A-2822E in the sample 2820. For the conventional flow cytometer, the system calculates the compensated event vector based on the initial spillover matrix and the measured sample event vector. For the spectral flow cytometer, the system calculates the unmixed sample event vector based on the initial reference matrix and the measured sample event vector. Additional steps can be taken to obtain even more accurate results using the initial spillover matrix and a reference matrix.

### Full Spectrum Flow Cytometer

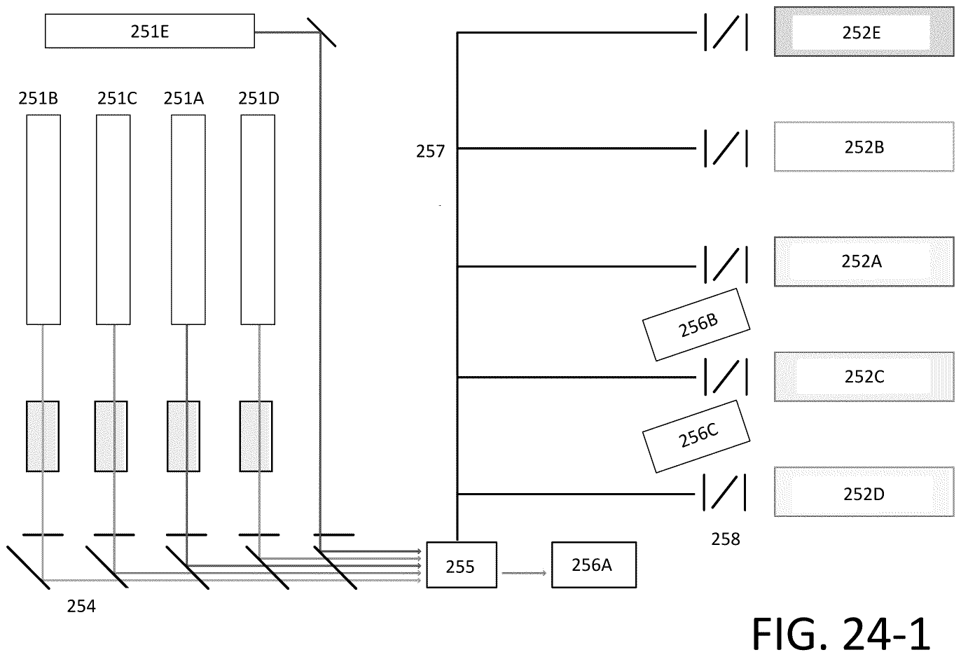

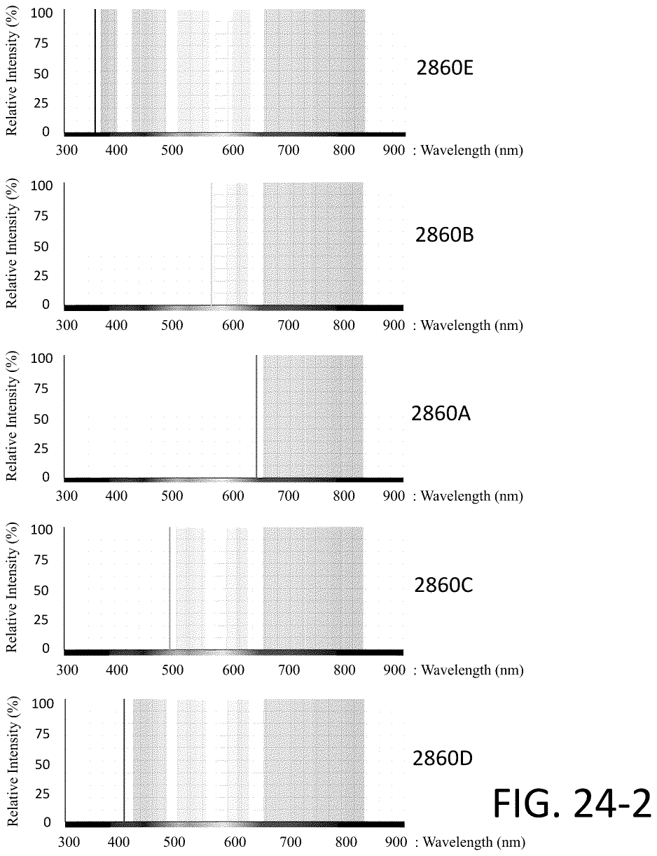

Referring now to FIG. 24 (FIGS. 24E-1 and 24-2), a schematic diagram of a full spectrum flow cytometer 2850 is shown. U.S. patent application Ser. No. 15/659,610 titled COMPACT DETECTION MODULE FOR FLOW CYTOMETERS filed on Jul. 25, 2017, by inventors Ming Yan et al., and U.S. patent application Ser. No. 15/498,397 titled COMPACT MULTI-COLOR FLOW CYTOMETER filed on Apr. 26, 2017, by David Vrane et al. describes further details of flow cytometers and are incorporated herein by reference.

The full spectrum flow cytometer 2850 can be variably configured with different numbers of lasers and different numbers of detector modules. In one embodiment, the full spectrum flow cytometer 2850 can include five lasers (Red 640 nm, Yellow-Green 561 nm, Blue 488 nm, Violet 405 nm, and UV 355 nm) 2851A-2851E and five detector modules 2852A-2852E as shown in FIG. 24-1 to provide full spectrum analysis. With five detector modules, each of the detector modules (Red, Yellow-Green, Blue, Violet, and UV) 2852A-2852E can be associated with one of the five lasers as shown in FIG. 24-1. Each of the five lasers generate laser light of five different wavelengths such as ultraviolet (UV) 355 nm, Violet 405 nm, Blue 488 nm, Yellow Green 561 nm, and Red 640 nm. Equipped with five lasers and five detectors, the full spectrum flow cytometer 2850 can be used to develop color panels with 28 or more colors.

The optical paths of the laser light for each of the five lasers (UV 355 nm, Violet 405 nm, Blue 488 nm, Yellow Green 561 nm, and Red 640 nm) is shown in FIG. 24-1. The lasers are spatially separated, each having an independent optical path to the flow cell 2855. One or more optical components 2854, such as mirrors, lenses, and filters, can be used to direct the laser light of each laser into the flow cell 2855 to strike particles/cells in the sample fluid as they pass by an interrogation region.

After striking a particle in the flow cell 2855, the fluorescent light is collected and directed through a plurality of optical fibers 2857 and one or more optical elements (e.g., lenses) 2858 into each of the individual detector modules 2852A-2852E. Each of the detector modules 2852A-2852E uses a sequential array of a plurality of avalanche photodiodes (APD) as the photodetectors. The full spectrum flow cytometer 2850 can further include a plurality of scatter detectors, including a forward scatter (FSC) detector 2856A near the flow cell, a blue side scatter detector 2856B near the lens/filters for the red detector module, and a violet side scatter detector 2856C near the lens/filters for the blue detector module. The plurality of scatter detectors are typically used to control data capture by the detector modules in the flow cytometer and data storage in a storage device. Each of the detector modules 2852A-2852E can capture a plurality of raw digital data for a given particle/cell as each laser beam of the plurality of lasers strike the same particle. The plurality of raw digital data is captured at slightly different times (laser delay) as the marked particle/cell passes by each laser beam in the flow channel. For example, the yellow/green laser may first strike the particle generating a first set of raw digital data, the violet laser second generating a second set of raw digital data, the blue laser third generating a third set of raw digital data, the red laser fourth generating a fourth set of raw digital data, and the UV laser lastly generating a fifth set of raw digital data for the same particle. If the plurality of lasers are arranged in a different order along the flow channel, the sequential order of generation of raw digital data by the same particle will be different. While an associated detector module is capturing light from its associated lasers, data from detectors in the other detector modules can be ignored. For example, at the time when the red laser strikes the particle/cell, the data from the red detector module is captured while the data from the UV, violet, yellow green, and blue detector modules can be ignored.

With the addition of the UV laser 2851A and having five detector modules providing sixty-four(64) fluorescence detectors (see FIG. 24E), the full spectrum flow cytometer 2850 has the power to take highly multiplexed assays beyond thirty (30) colors. The incorporation of the UV laser 2851A allows the full spectrum flow cytometer 2850 to perform at a different wavelength and discriminate different colors than those systems without. The UV laser enables the use of UV light excited fluorochromes, such as BUV737 and BUV395 fluorochromes, giving researchers additional flexibility on how they design experiments for a sample of particles.

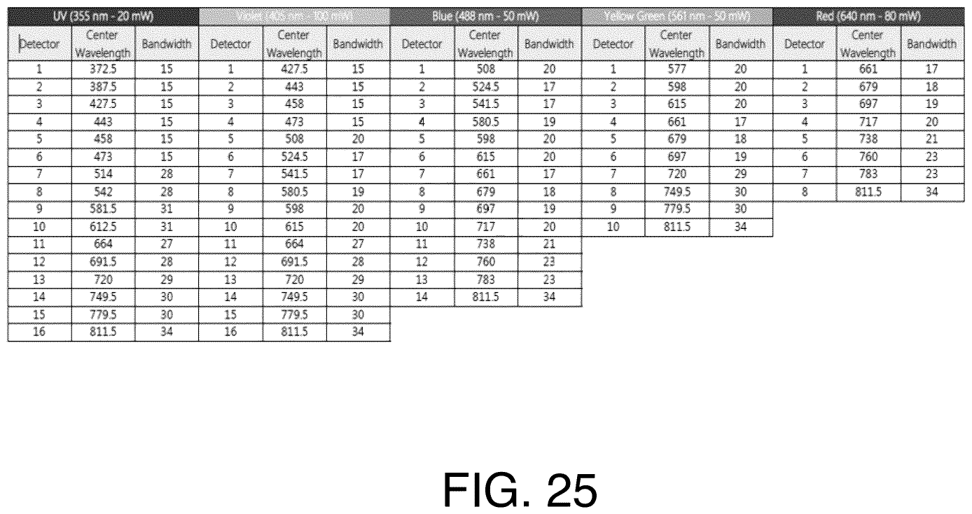

FIG. 25 illustrates the configuration of each photodetector in each of the five detector modules 2852A-2852E used in the embodiments of a full spectrum flow cytometer 2850. Each detector has a bandpass filter in front of it to filter out light. The bandpass filter allows predetermined wavelengths through to the photo detector for detection while filtering out other wavelengths. The detector number (also referred to herein as channel number) and wavelength information of the bandpass filters associated with each photo-detector is shown. The ultraviolet (UV) detector module 2852E has sixteen (16) detectors labeled as channels UV1-UV16 based on their position in the sequential array of detectors in the module. The violet detector module 2852D has sixteen (16) detectors labeled as channels V1-V16 based on their position in the sequential array of detectors in the module. The blue detector module 2852C has fourteen (14) detectors labeled as channels B1-B14 based on their position in the sequential array of detectors in the module. The yellow green detector module 2852B has ten (10) detectors labeled as detector channels YG1-YG10 based on their position in the sequential array of detectors in the module. The red detector module 2852A has eight (8) detectors labeled as detector channels R1-R8 based on their position in the sequential array of detectors in the module.

The multiple lasers in the flow cytometer are slightly spaced apart and sequentially strike the same particle/cell as it flows through the flow channel. This sets up a small amount of time delay between each subsequent laser strike (laser intercept) of the same particle/cell. There is a similar amount of time delay in the respective signal detected by the detectors and the generation of digital data from each laser strike (laser intercept) for the same particle/cell. The small amount of time is referred to as laser delay time and is predetermined by running a quality control experiment (e.g., daily QC runs) before running an experiment with a biological sample or other control. The full spectrum of fluorescence light from each laser striking the particle/cell is sent to each detector module by the fiber optic cables 2857. Based on the laser delay time, the data generated by the detectors from each laser strike (laser intercept) can be associated with a given laser. For example, at one point in time a blue laser strikes the particle/cell and the detectors in the blue detector module can detect fluorescence and generate data for the blue laser strike. After a predetermined laser delay time between blue and red lasers, the same particle is struck by the red laser. Based on the time of the red laser strike, the detectors in the red detector module can detect fluorescence and generate data associated with the red laser strike. The laser delay time between the different lasers can be different but predetermined in order to be able to associate the captured data with the appropriate laser. Furthermore, the arrangement of the lasers can be in a different sequential order such that the sequence of laser strikes can differ. Moreover, the associated laser delay time can differ between laser strikes between power cycles of the flow cytometer. In any case, the data generated by each respective module that is delayed from the first data generated, is aligned together in time and associated with the particle/cell of a single event. The captured data from each detector module may be tagged with a particle/cell number count in the sample run and temporarily stored in a storage device, such as a register, memory or hard drive, for subsequent alignment together as a single event.

Fluorochromes are excited over a wavelength range (excitation wavelength range) associated with the wavelength of the laser and when excited, can emit fluorescence over a different wavelength range (emission wavelength range). The wavelength range of each detector module is associated with the expected emission wavelength range from the excitation of fluorochromes for the associated laser.

With reference to FIG. 25, the bandpass filter before each detector is used to selectively pass the desirable wavelengths in the pass band range to be detected at a given photo detector for the associated excitation laser. The band bass filter rejects the wavelengths of light outside the pass band range of wavelengths. For example, the first red detector channel (R1 detector channel), the band pass filter has a center wavelength of 661 nanometers (nm) and a bandwidth of 17 nanometers around the center wavelength. Accordingly, in the band pass of wavelengths, a detector can reliably detect a wavelength range around a center wavelength and plus and minus one half the bandwidth. In the case of the R1 detector channel shown in FIG. 25, the wavelength range is from the center wavelength minus one half the bandwidth (661 nm 8.5 nm=652.5 nm) to the center wavelength plus one half the bandwidth (661 nm+8.5 nm=669.5 nm). In the case of the R8 detector channel, the wavelength range is from the center wavelength minus one half the bandwidth (811.5 nm−17 nm=794.5 nm) to the center wavelength plus one half the bandwidth (811.5 nm+17 nm=828.5 nm). Accordingly, the red detector module detects fluorescent light over a wavelength range from 625 nm to 828.5 nm for fluorescent particles excited by the red laser. The yellow green detector module detects fluorescent light over a wavelength range from 567 nm to 828.5 nm for fluorescent particles excited by the yellow green laser. The blue detector module detects fluorescent light over a wavelength range from 498 nm to 828.5 nm for fluorescent particles excited by the blue laser. The violet detector module detects fluorescent light over a wavelength range from 420 nm to 828.5 nm for fluorescent particles excited by the violet laser. The ultra violet detector module detects fluorescent light over a wavelength range from 365 nm to 828.5 nm for fluorescent particles excited by the ultra violet laser. This detection range includes the full visible light (electromagnetic) spectrum from 380 nm to 780 nm, a portion (365 nm to 379 nm) of the non-visible UV light spectrum, and a portion (781 nm to 828.5 nm) of the non-visible infrared light spectrum.

If even more than 64 detectors are used, an increased granularity in the data at various wavelengths can be captured. The compactness of photo detectors (e.g., avalanche photo-diodes) and the detector array in the detector module has led to embodiments of up to 64 detectors and can lead to a further increase in the numbers of available detectors. A larger number of detectors can lead to increased numbers of colors that can be detected (discriminated) and an increased number of fluorochromes that can be used to examine particles within a single sample by a single run through a flow cytometer. The use of compact photodetectors in a compact photo detector array as the detector modules in the full spectrum flow cytometer 2850 has improved the efficiency of running samples through a flow cytometer and examining the resultant data.

While a single particle has been described passing through each laser, a sample fluid run through a flow cytometer can have thousands of cells/particles per micro liter with hundreds of thousands or more of particles in a sample fluid size of hundreds of microliters (e.g., 500,000 particles in a 500-microliter sample size). The same sample can have different types of cells with hundreds of thousands or more. With a multi-color experiment, different fluorochromes are attached to different particles/cells to count different types of particles in the same sample. In a single run through the flow cytometer, the intensity and wavelength of each color of fluorescent light generated by the excited fluorochrome on the labeled cells can be detected and plotted on a chart by wavelengths to indicate the spectrum of light captured by the sample run. Furthermore, the intensity of fluorescent light for each given color/detector channel can be binned into count ranges with the particle count falling into these ranges being summed up together and plotted on the chart to show the particle cell density for the wavelengths of light.

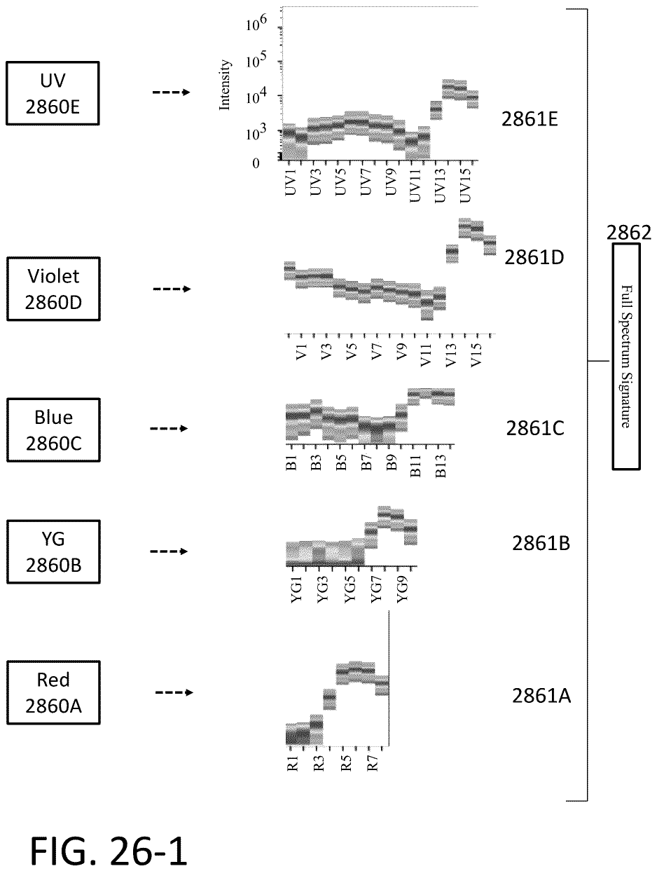

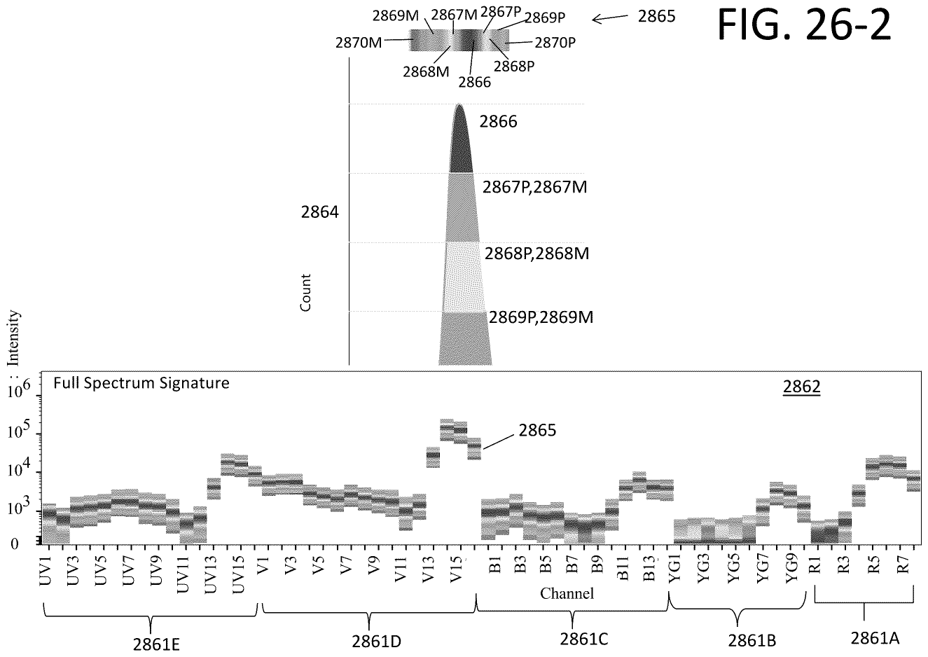

In FIG. 24-2, the charts 2860A-2860E of data, normalized intensity (Y axis) versus wavelength (X axis), represents the range of light spectral components captured by each respective detector module for all events (each cell passing through the lasers) in a sample, such as a reference control with a single fluorochrome being used to generate a reference full spectrum signature. In FIG. 26-1, the raw channel data captured for each detector module 2852A-2852E can respectively be plotted, based on the detector channel number, as a portion (individual detector module spectrum signature) 2861A-2861E of a full spectrum (spectral) signature of the sample run. In the plots of the individual detector module spectrum signature portions 2861A-2861E associated with each color laser 2851A-2851E and associated detector module 2852A-2852E pairing, the intensity (Y axis) and binned density count are plotted as a function of the detector channel number (X axis). Each of the individual detector module spectrum (spectral) signatures is formed out of a channel spectrum signature, such as channel spectrum signature 2865 for the detector module spectrum (spectral) signature 2861D for example.

The channel spectrum signature is plotted based on a plurality of binned intensity levels and the particle counts within those bins. For example, the greatest count (highest density) at the binned intensity level range for the channel is given a first color (e.g., red) located at the center intensity level range 2866 of the channel spectrum signature 2865. For each channel spectrum signature, the other binned intensity levels are either above 2867P,2868P,2869P or below 2867M,2868M,2869M the center intensity level 2866 having the greatest particle/cell count. The second intensity levels 2867P,2867M respectively just above 2867P and below 2867M the center intensity level 2866 are assigned a second color differing from the first color of the center intensity level. The third intensity level 2868P above the second and center intensity levels and the third intensity level 2868M below the second and center intensity levels are assigned a third color differing from the first and second colors. The fourth intensity level 2869P above the third, second, and center intensity levels and the fourth intensity level 2869M below the third, second and center intensity levels are assigned a fourth color differing from the first, second, and third colors. In this manner, intensity density information can be communicated to the user for a given detector channel.

After generating plots of the individual detector module spectrum (spectral) signatures 2861A-2861E, the plots of the individual detector module spectrum (spectral) signatures can then be merged together. In FIG. 26-2, the individual detector module spectrum (spectral) signatures 2861A-2861E are merged together along an X axis of detector channel number to form a plot of a full spectrum (spectral) signature 2862 of the exemplary sample run through the full spectrum flow cytometer. Along the X axis, from right to left, are the red detector module spectrum signature 2861A, the yellow green detector module spectrum signature 2861B, the blue-detector module spectrum signature 2861C, the violet detector module spectrum signature 2861D, and the ultraviolet detector module spectrum signature 2861E merged together forming the full spectrum signature for a given sample run. Different labeled samples run through the flow cytometer 2850, will generate different detector module signatures and accordingly different merged full spectrum (spectral) signatures. Single stained control samples (reference controls) are run through the full spectrum flow cytometer used to determine the full spectrum signature of each fluorochrome before being used with other fluorochromes to label a particle/cell in a mixed sample of a plurality of particles/cells.

Instead of just looking at peak intensity levels, the full spectrum signature for one fluorochrome can be used to distinguish from noise and another fluorochrome having a different full spectrum signature. Detecting light intensity over the full spectrum is an advantage of a full spectrum flow cytometer over that of a conventional flow cytometer that just looks at peak intensity levels. When a conventional flow cytometer shows overlap in the spectrum plots of fluorescent dies, the full spectrum signatures of each when run through a full spectrum flow cytometer can be distinguishable. In planning an experiment, it is desirable to select different fluorochromes that can be distinguishable from each other by their full spectrum signatures. Fluorochromes with similar emission but different spectral signatures can be distinguished from each other. The mathematical method to differentiate between multiple fluorophores (mixed fluorescent light) is called spectral unmixing and results in an unmixing matrix that is applied to the captured data of the sample.

Particles/cells may auto-fluoresce (autofluorescence) when struck by the five lasers and have its own full spectrum signature. Accordingly, the autofluorescence of the various particles/cells can also be unmixed, based on the autofluorescence full spectrum signature, and be used to distinguish it from other particle/cell types and the fluorochrome attached to other cells in a mixed sample.

While certain exemplary embodiments have been described and shown in the accompanying drawings, it is to be understood that such embodiments are merely illustrative of and not restrictive on the disclosed embodiments, and that the disclosed embodiments are not limited to the specific constructions and arrangements shown and described, since various other modifications may occur to those ordinarily skilled in the art.

While this specification includes many specifics, these should not be construed as limitations on the scope of the disclosure or of what may be claimed, but rather as descriptions of features specific to particular implementations of the disclosure. Certain features that are described in this specification in the context of separate implementations may also be implemented in combination in a single implementation. Conversely, various features that are described in the context of a single implementation may also be implemented in multiple implementations, separately or in sub-combination. Moreover, although features may be described above as acting in certain combinations and even initially claimed as such, one or more features from a claimed combination may in some cases be excised from the combination, and the claimed combination may be directed to a sub-combination or variations of a sub-combination. Accordingly, the claimed invention is limited only by patented claims that follow below.

1-89. (canceled)

90. A method for a fluorescent non-conjugated polymeric tandem dye nanoparticle, the method comprising:
forming a non-conjugated polymer backbone with side chains by
copolymerizing reactive monomers connected to fluorescent donor monomers, FRET monomers, acceptor dyes, and functional groups;
forming nanoparticles of the non-conjugated polymer backbone and sidechains by
injecting a solution of the non-conjugated polymer backbone with side chains into water under sonification; and
bioconjugating the nanoparticle with an antibody by
PEGylating the nanoparticle to react to a sulfhydryl group on the antibody, and
energizing the fluorescent donor monomers with a laser and detecting fluorescence of the acceptor dye in a full spectrum flow cytometer.

91. A method for producing inorganic nanoparticle fluorescent dye complexes for flow cytometry and biological applications, the method comprising:
a) hydrolyzing a functional coupling agent to cover reactive functional groups on a surface of a plurality of inorganic nanoparticles;
b) reacting fluorescent dyes having functional groups with functionalized inorganic nanoparticles;
c) reacting linker molecules or oligomers with functional groups on the surface of the plurality of inorganic nanoparticles firstly; and
d) directly bio-conjugating functionalized fluorescent inorganic nanoparticles with antibodies or other bioactive molecules.

92. The method of claim 91, further comprising:
reacting linker molecules or oligomers with a plurality of antibodies or other bioactive molecules to activate the plurality of antibodies or other bioactive molecules.

93. The method of claim 92, further comprising:
directly bio-conjugating functionalized fluorescent inorganic nanoparticles with activated antibodies or other bioactive molecules.

94. The method of claim 91, wherein:
the inorganic nanoparticles are metal oxide nanoparticles.

95. The method of claim 94, wherein:
the inorganic nanoparticles include alumina nanoparticles, silica nanoparticles, titania nanoparticles, indium tin oxide nanoparticles, zinc oxide nanoparticles, iron oxide nanoparticles, antimony tin oxide nanoparticles, or nanoparticles covered with an inorganic metal oxide layer.

96. The method of claim 91, wherein:
the size of the inorganic nanoparticles is less than 500 nanometers, 200 nanometers, 100 nanometers, 50 nanometers, 25 nanometers, 15 nanometers, 10 nanometers, or 5 nanometers.

97. The method of claim 91, wherein:
the functional coupling agent is one of a silane coupling agent, a titanate coupling agent, an aluminate coupling agent, a zirconate coupling agent, a phosphate coupling agent, and a borate coupling agent.

98. The method of claim 91, wherein:
the functional organic groups on the functional coupling agents include one or more of alkylhalide, azide, amino, alkyne, aldehyde, maleimide, hydroxyl, acetal, isocyanate, epoxide, acrylate, sulfonate (tosyl, mesyl), nitrophenyl carbonate, Biotins, folic acid, methacrylate, mercapto, tetrafluorophenyl esters, succinimidyl ester, pentafluorophenyl ester, hydrazides, vinyl, vinylsulfone, dibenzocyclooctyne group (DBCO), and methyltetrazine.

99. The method of claim 91, wherein:
the linker that connects the functional groups to a silicon atom includes one of an alkyl chain, a peptide chain, and a polyethylene oxide chain.

100. The method of claim 91, wherein:
the number of repeated units of the linker ranges from 5,000 to 1, from 3,000 to 1, from 2,000 to 1, from 1,000 to 1, from 500 to 1, from 100 to 1, or from 20 to 1.

101. The method of claim 91, wherein:
the fluorescent dye is a fluorescent chemical compound that can emit light upon laser excitation.

102. The method of claim 98, wherein:
the fluorescent dye is at least one of BODIPY derivatives, dipyrrin-metal derivatives, Atto derivatives, Cyanine derivatives, squaraine derivatives, Fluorescein derivatives, porphyrin, metalloporphyrin derivatives, phthalocyanine derivatives, Rhodamine derivatives, lanthanide complexes derivatives, and Pyrene dyes.

103. The method of claim 98, wherein:
the fluorescent dye is an organic fluorescent dye with a narrow bandwidth of light absorption between 260 nanometers and 900 nanometers and a narrow bandwidth of fluorescence between 260 nanometers and 1100 nanometers.

104. The method of claim 91, wherein:
the fluorescent dye has functional groups that can react with functional groups on a surface of inorganic nanoparticles.

105. The method of claim 104, wherein:
the functional groups on the fluorescent dye is at least one of an amino, an alkylhalide, an azide, an alkyne, an aldehyde, a maleimide, a hydroxyl, an acetal, an isocyanate, an epoxide, an acrylate, a sulfonate (tosyl, mesyl), a nitrophenyl carbonate, a Biotins, a folic acid, a methacrylate, a mercapto, a tetrafluorophenyl ester, a succinimidyl ester, a pentafluorophenyl ester, a hydrazides, a vinyl, a vinylsulfone, a dibenzocyclooctyne group (DBCO), and a methyltetrazine and other reactive functional organic groups.

106. The method of claim 104, wherein:
the functional groups on the fluorescent dye are reactive functional organic groups.

107. The method of claim 91, wherein:
the fluorescent dyes react with functional groups on surface of inorganic nanoparticle by a condensation reaction, a click chemistry reaction, a photochemistry reaction, a Suzuki coupling reaction, a Stille coupling reaction, a Sonogashira coupling reaction; or a Heck, Mcmurray and Knoevenagel, Wittig, Horner reaction.

108. The method of claim 91, wherein:
the linker molecules or oligomers with functional groups are an oligomer chain with one functional group, an oligomer chain with two functional groups, or branched oligomers with multi-functional groups.

109. The method of claim 91, wherein:
the functional groups in linker molecules or oligomers include one or more of an amino, an alkylhalide, an azide, an alkyne, an aldehyde, a maleimide, a hydroxyl, an acetal, an isocyanate, an epoxide, an acrylate, a sulfonate (toys, mesyl), a nitrophenyl carbonate, a Biotins, a folic acid, a methacrylate, a mercapto, a tetrafluorophenyl ester, a succinimidyl ester, a pentafluorophenyl ester, a hydrazides, a vinyl, a vinylsulfone, a dibenzocyclooctyne group (DBCO), an a methyltetrazine.

110. The method of claim 91, wherein:
the functional groups in linker molecules or oligomers are reactive functional organic groups.

111. The method of claim 91, wherein:
the backbone of the linker molecules are one of an alkyl chain, a peptide chain, and a polyethylene oxide chain.

112. The method of claim 111, wherein:
the number of repeated units in the linker molecules range from 10,000 to 1, from 5,000 to 1, from 3,000 to 1, from 2,000 to 1, from 1,000 to 1, from 500 to 1, from 100 to 1, or from 20 to 1.
